(19) United States
(12) Patent Application Publication
(10) Pub. No.: US 2026/0241343 A1
(43) Pub. Date: Aug. 20, 2026

(54) METHOD FOR TESTING THE INTEGRITY OF VIRUS FILTRATION MODULES AND A FILTRATION APPARATUS FOR CARRYING OUT THE METHOD

(71) Applicant: Boehringer Ingelheim International GmbH, Ingelheim am Rhein (DE)

(72) Inventors: Raiko Seifert, Attenweiler (DE); Christian KLEY, Wien (AT); René Robert SCHULER, Oberdingen (DE)

(21) Appl. No.: 19/162,611

(22) PCT Filed: Mar. 7, 2024
(86) PCT No.: PCT/EP2024/055973

§ 371 Date: Sep. 5, 2025

(30) Foreign Application Priority Data
Mar. 8, 2023 (EP) 23160635.1

(51) Int. Cl. B01D 65/10; B01D 63/02; C07K 16/00

(52) U.S. Cl. CPC B01D 65/104; B01D 63/02; C07K 16/00; B01D 2313/60; B01D 2313/903; B01D 2317/04

(57) ABSTRACT

The disclosure relates to a method for testing the integrity of one or more virus filtration modules and to a filtration apparatus for carrying out the method, by means of which the step of detecting whether an intact virus filtration module is present or not can be carried out in a fully automated manner for the first time. Multiple virus filtration modules can be tested in parallel at the same time, thus resulting in a distinct time saving when testing. The automation of detection allows reliable testing, wherein test results are reproducible and the risk of damage to the hollow fibers during testing is minimized. The integrity test can be carried out in the very same filtration apparatus in which virus filtration with the tested virus filtration modules can also be effected between the integrity tests, without the need to install or remove the virus filtration module(s) in a separate test system.

### BACKGROUND OF THE INVENTION

### Technical Field

The present disclosure relates to a method for testing the integrity of virus filtration modules and a filtration apparatus for carrying out the method.

### Prior Art

Filters are of great importance for a wide range of technical applications. For example, in the aseptic production of pharmaceuticals or medical products, so-called sterile filters are used to prevent contamination by, e.g., germs or particles by separating them. It has been common practice for years, in accordance with GMP (Good Manufacturing Practice) guidelines and regulations issued by the EMA (European Medicines Agency) and the FDA (U.S. Food and Drug Administration), to test filters for integrity prior to and/or after use (“post-use filter integrity” and “pre-use filter integrity”). The integrity test is a functional test of the filter intended to demonstrate that the filter was or is fully operational and thus intact, i.e., does not allow any undesirable components to pass through. Known integrity tests are destructive or non-destructive. Non-destructive integrity tests are generally preferred, as they are more advantageous for economic reasons.

Non-destructive integrity tests known in the pharmaceutical sector include, for example, the bubble point test, the diffusion test (forward flow test and pressure hold test) and the water flow test, wherein, in each case, the manufacturer's guidelines for the filters can be used as a guideline in order to check whether the filters are actually functional.

Membrane filters are used, for example, in biotechnology, pharmaceuticals and food technology. The separation method is effected purely mechanically via the membrane of the filter, does not require heating like conventional separation methods, such as distillation, and therefore represents a gentle separation. Membranes also make separations possible that cannot be achieved using conventional thermal separation methods; moreover, the components being separated are not damaged, e.g., by thermal effects.

Particularly in biopharmaceutical manufacturing processes and in view of the global Covid pandemic, the removal of viruses with the aid of filter membranes has gained importance. The presence of viruses, for example in medicines and medical products, poses a significant risk to a patient of viral infection, if applicable even with the potential danger of spreading the infection. As is well known, virus filters are used for this purpose, which are able to completely separate and thus remove viruses from a batch.

Typical virus filters are, for example, composite membranes, which can be made of a plurality of materials. For example, a support structure made of fibers and a fine-pored polymer support layer is combined with an extremely thin, pore-free separating layer. Virus filters are usually virus retention filters in the form of ultra- or microfilters, which have very small and additionally defined pore sizes. The filters are differentiated according to the size of the viruses to be removed. For example, retrovirus filters are known, wherein the diameter of a retrovirus is approximately 80 to 100 nm, and parvovirus filters, wherein parvoviruses have a diameter of approximately 18 to 26 nm.

Other well-known virus filters are so-called hollow fiber filters, which are a bundle of fine tubes with small pores. Hollow fiber filters are used to filter liquids and usually have two inlets and two outlets. Virus removal is based on a combination of size exclusion and depth filtration mechanisms, wherein viruses larger than the mean pore size are retained by the hollow fibers. Such hollow fiber filters are used, for example, to remove viruses from pharmaceuticals such as biopharmaceuticals and plasma derivatives.

For a better understanding, hollow fiber filters and their function are initially explained with reference to the figures, before the method known from the prior art for testing the integrity of hollow fiber filters is described:

FIG. 1 is a schematically simplified view of a hollow fiber filter module 10 without a usually present housing, which surrounds the filter module 10, so that the hollow fibers can be seen inside the filter module 10. In detail, a bundle 50 of hollow fibers is shown, which is composed of a plurality of hollow fibers 50.1, 50.2, . . . The hollow fibers 50.1, 50.2, . . . are hollow in the inner region 35, also referred to as the hollow fiber inner side, and are in the form of membrane tubes with small diameters, which are arranged substantially parallel to one another. The hollow fibers 50.1 and 50.2 have a lower end 20 to which liquid is fed (so-called feed, arrow A) and an upper end 30 at which rinsing solution emerges from the hollow fibers 50.1 and 50.2 only during a rinsing process of the filter module 10 (arrow B), but no liquid emerges during filtration. The wall 40 of the hollow fibers 50.1, 50.2, . . . in each case has pores (not shown) so that liquid passes through the wall 40 of each of the hollow fibers 50.1, 50.2, . . . (arrow C). Therefore, the hollow fiber walls act as a filter. The components retained in the inner region 35 or on the inner side or lumen of the hollow fibers 50.1, 50.2, . . . are also referred to as retentate; the filtered liquid passing through the walls of the hollow fibers 50.1, 50.2, . . . is also referred to as filtrate. Therefore, the hollow fiber filter module 10 has a feed side 55, a retentate side 65 and a filtrate side 75. The feed side 55 leads to the inner side or inner region 35 of each hollow fiber. Between the hollow fibers 50.1, 50.2 . . . , there are interstices 38.1, 38.2 . . .

FIG. 2 is a schematic enlarged sectional view of a hollow fiber 50.1, which is present in the hollow fiber filter module 10 shown in FIG. 1 as part of the hollow fiber bundle 50. If, for example, a protein solution on the feed side 55 with viruses 100.1, 100.2, . . . is introduced as contamination into the interior 35 of the hollow fiber 50.1 as symbolized by arrow A-the protein solution penetrates the pores (e.g., pore 45) of the membrane wall 40 on the filtrate side 75 to the outside, as symbolized by the arrows C (downward and upward). The pores in the wall 40 of the hollow fiber 50.1 are symbolized by the dashed lines in FIG. 2. The dense pore structure of the hollow fiber wall 40, for example, is several dozen micrometers thick and represents a kind of winding, three-dimensional network of interconnected cavities and capillary pores. The viruses 100.1, 100.2, . . . are effectively retained by the net-like pore structure in the hollow fiber wall 40, while the now virus-free protein solution with the proteins migrates outward through the hollow fiber wall 40 and is retained on the filtrate side 75 (arrows C). At arrow B, no liquid exits during filtration (symbolized by the crossed-out arrow B); only during a rinsing process in the integrity test does liquid exit, as will be explained.

The separation of viruses only functions if the hollow fiber filter module is fully functional and the hollow fibers are intact and show no damage of any kind. In order to detect whether the filter is intact, tests have been developed to check this. These are so-called integrity tests, i.e., tests that make it possible to detect whether the existing hollow fibers are undamaged and are functioning within the desired framework. This integrity test of the virus filter function along with further integrity tests not described here are a prerequisite for the release of a filtered product after completion of virus filtration. A further prerequisite for release for virus filtration is a gold particle (GPT) test, also an integrity test for a hollow fiber filter, which serves to confirm the filter quality (distribution of pore sizes) after filtration. The GPT test plays no role here and is not the subject matter of the present invention, which is why it will not be discussed in more detail here.

The integrity test of interest now shows whether the hollow fibers of the filter module are undamaged. The testing of the hollow fiber filter module is effected prior to use in order to ensure that the filter module is suitable for use and is functioning properly. In addition, a further integrity test is effected after use of the filter module, i.e., after virus filtration, in order to ensure that it has not been damaged during filtration, for example due to overpressure or mechanical stress. The integrity test, as known from the prior art, is substantially carried out as follows:

A hollow fiber filter module 10 usually has two inlets and two outlets. This is shown by way of example in FIG. 3a. FIG. 3a shows a schematically enlarged front view of a virus filtration module in the form of a hollow fiber filter module 10 with connected lines. There are two inlets at 55 and 65 and two outlets at 75o and 75u. However, for the integrity test, only one inlet, for example at 55, and one outlet, for example at 75o, are regularly used, to which the lines shown are connected in each case. The outlet at 75o in the filter module 10 may have advantages for method-related reasons, for example due to a short path to the assigned connector. The further entrance at 65 and the further exit at 75u are or will be closed. The two inlets at 55 and 65 in the filter module 10 are completely equivalent, so that it does not matter which of the two inputs is selected. This also applies to the outlets, which are both completely equivalent. In FIG. 3a, therefore, the inlet at 65 and the outlet at 75u could also be selected for the integrity test and the inlet at 55 and the outlet at 750 could be or become closed.

In the integrity test known in the prior art (see “TAS 33088, Integrity Test Procedure Planova™ 15N, 20N, 35N and 75N Filters, Visual Leakage Test (VLT),” version 1.1 dated 25.02.2019, the specification is available for download from the Asahi Kasai website after registration), the hollow fiber filter module 10 to be tested is initially placed in a vertical position and filled with liquid under overpressure. Then air is blown under pressure into the line on the inlet side at 55 (feed side) of the filter module 10. For this purpose, the lines on the inlet side at 55 and the outlet side at 75oof the filter module 10 are opened for the test setup and the further lines or their connections (at 65 and 75u) are or are already closed. The pressure is kept constant throughout the entire test. During the test period, a visual inspection is then carried out to check whether or not a continuous chain of air bubbles is detected in the line on the outlet side at 75o. If a chain of air bubbles occurs, this means that the hollow fiber filter module 10 is damaged and is not functioning to the desired extent.

The relationships that play a role in carrying out the integrity test on a hollow fiber in a virus filtration module are illustrated in detail below with reference to FIGS. 3b and 3c for better understanding:

In FIG. 3b, an intact hollow fiber 50.1a is shown schematically and simplified in an enlarged sectional view. The hollow fiber 50.1a is part of a hollow fiber bundle 50 as illustrated in FIG. 1, which is located with other hollow fibers in a housing (not shown). The hollow fiber filter module has already been completely filled with liquid according to the integrity test to be carried out, so that there is liquid in the entire filter module i.e., in the hollow fibers and in the interstices between the hollow fibers-as well as in all lines leading to and from the filter module. In FIG. 3b, therefore, there is liquid in the inner region 35 of the hollow fiber 50.1a and there is also liquid outside the wall 40 of the hollow fiber 50.1a in the interstice between the hollow fibers. The volume of the existing liquid is delimited by the type and size of the hollow fiber filter module and its housing along with the existing lines.

As can be seen from FIG. 3b, air 80 is fed under pressure from the feed side 55 into the inner region 35 or the inner side of the hollow fiber 50.1a (arrow A). In FIG. 3b, the hollow fiber 50.1a shown is intact and exhibits no damage. In this case, the air 80 introduced from the feed side 55 passes through the hollow fiber 50.1a and only a very small amount of air passes through the membrane. This small amount is also referred to as the “leak flow” and will be explained in more detail later. Therefore, the air 80 does not leave the interior 35 of the hollow fiber 50.1a, since the air 80 cannot pass through the pores in the wall 40 of the hollow fiber 50.1a, but is retained in the interior 35 together with the existing liquid. The pores in the wall 40 of the hollow fiber 50.1a are symbolized by the dashed lines in FIG. 3b. Thus, practically no fed air 80 exits the hollow fiber 50.1a on the filtrate side 75 (arrows C). The hollow fiber 50.1a of FIG. 3b is therefore an intact hollow fiber 50.1a, which would also pass the integrity test.

Unlike in FIG. 3b, in FIG. 3c a damaged hollow fiber 50.1b is shown schematically and simplified, in an enlarged sectional view. There is damage 90 in the wall 40 of the hollow fiber 50.1b. The damage 90 shown represents, for example, a crack or other impairment of the pore structure of the hollow fiber wall 40. As a result, the introduced air 80 cannot be retained in the interior 35 of the hollow fiber 50.1b, but rather exits to the outside on the filtrate side 75 through the wall 40 of the hollow fiber 50.1b. Therefore, the occurrence of a chain of air bubbles in the filtrate would indicate damage to the hollow fiber 50.1b, which can then no longer be used. If such a damaged hollow fiber 50.1b were to be used in a virus filtration, the potentially present viruses would also no longer be retained in the interior 35 of the hollow fiber 50.1b and would exit through the damage 90 and continue to contaminate the product to be purified. Separating viruses would no longer be possible reliably.

Due to the undesirable exiting of air through the hollow fiber wall and the assessment of the presence of a chain of air bubbles by visual inspection, the test is also referred to as the “visual leak test,” which is effected on a virus filter prior to carrying out filtration (pre-visual leak test) and also after filtration (post-visual leak test). A visual leak test or integrity test is only considered passed if no continuous chain of air bubbles is detected for a duration of at least 20 seconds over the entire test period.

The general procedure and test parameters of the integrity test were designed by Asahi Kasai and contain the test setup, wherein air flows through a hollow fiber module at a defined pressure and potential damage caused by a continuous chain of air bubbles at the outlet of the filter module is detected through a manual and purely visual inspection by one or more employees (see “TAS 33088, Integrity Test Procedure Planova™ 15N, 20N, 35N and 75N Filters, Visual Leakage Test (VLT),” version 1.1 dated 25.02.2019; the specification is available for download from the Asahi Kasai website after registration).

The well-known integrity test, as described above, has a number of serious disadvantages:

A disadvantage of the well-known integrity test is that the hollow fiber filter modules used to separate viruses are highly sensitive and can be easily damaged if handled improperly, thus destroying their function. This is particularly a problem in the test setup for the integrity test, if the hollow fiber filter modules have to be installed and removed several times for testing the integrity in the test apparatus, in order to test the function of the hollow fiber filter modules prior to and after virus filtration. A further problem arises if unsuitable or incorrect test parameters that can impair the filter are selected for the test. These are potential risks for possible damage to the hollow fiber filter modules. If damage is detected after virus filtration, it is always difficult to detect retrospectively whether damage already existed prior to filtration, occurred during filtration or was caused by the integrity test after filtration. However, sterility, in particular freedom from viruses, is absolutely essential in specific technical areas, for example in biopharmaceutical purification processes.

Another problem is that the visual inspection to detect whether and to what length the air bubbles occur and indicate a possible defect in the hollow fibers is effected by production employees. This represents a subjective evaluation of the test, wherein the specification of and ability to recognize a chain of air bubbles is not clearly defined and therefore not subject to clear measurable acceptance criteria. For example, one observer sees a continuous chain of air bubbles for at least 20 seconds, while another observer sees or interprets this differently. Thus, the results depend on the respective observer and are not objective. Even if a plurality of independent observers carry out the visual inspection, this does not really improve the results, since human error in the setup of the test and carrying out cannot be ruled out. In fact, it would only tie up more employee capacity for testing. In addition, the test results are often not reproducible when repeated. Furthermore, the test procedure and the result cannot be recorded; rather, only the number of observers increases (e.g., double-checked) and the result cannot be documented accordingly. Therefore, the test result is purely subjective and is determined only visually. The test evaluation is potentially prone to errors due to the absence of clearly defined evaluation parameters.

In addition, the known methods from the prior art usually only capture medium and large defects, but not small and very small defects, such as fine cracks.

Some suggestions for carrying out integrity tests have emerged from the prior art:

For example, FR 2 909 904 A1 describes a method for checking the integrity of hollow fiber type filter membranes, wherein a static pressure difference is applied between the two compartments of a hollow fiber membrane, in particular by pressurizing one of the two compartments (external or internal) with the aid of a gas that has been previously emptied of the water it contains (emptied compartment). The other compartment remains filled with water (water compartment). The pressure is selected to be lower than the bubble pressure of the membrane but higher than the pressure of the water compartment. By way of example, a pressure difference of 0.5 bar=50 kPa is specified. If a membrane is not intact due to a rupture of the hollow fiber, air flows in the form of bubbles through the broken fiber from the emptied compartment into the water compartment due to the pressure difference on both sides of the membrane. The checking of the integrity is effected by measuring the dynamic overpressure that is generated by the air bubbles in the water chamber. Filtration serve in particular for water treatment, wherein the sensitivity depends on the quality of the raw water used. The hollow fiber membranes used should also be suitable for microfiltration, ultrafiltration, nanofiltration or reverse osmosis. However, in FIGS. 1 and 2, an inner diameter in the millimeter range is specified for the inner channels of the hollow fibers. In contrast, viruses have diameters in the nm range, so the focus here is on larger regions. A step-by-step filling of the hollow fiber membranes is not described, so that they cannot be completely filled and the reliability of the integrity test is not ensured. The volume flow of the gas through the hollow fiber membranes is also not measured, so that the recognition of even the finest defects is not possible.

JP 2005 013947 A provides a membrane separation apparatus that can be used to specify damaged hollow fibers during a water circulation process. For this purpose, bubbles are mixed into the raw water. If a hollow fiber membrane is damaged, bubbles enter the hollow fiber membrane 4 and flow out of an opening 4a at the upper end into a permeation space 14. In addition, fine powder exiting the defect of the hollow fiber membrane, which is dispersed in the gas, is used for recognizing the defect of the hollow fiber membrane. The bubbles exiting the opening 4a are recognized based on the images of a CCD camera 24, as a result of which the position of the damaged hollow fiber membrane is determined. Raw water is processed, so that this system cannot be used for virus filtration. Only large defects are recognized, since only in this case does fine powder occur. There is no step-by-step filling of the membrane separation apparatus, so a reliable integrity test cannot be effected. In addition, the volume flow of the gas through the membrane separation apparatus is not measured, so that the recognition of even the finest defects is not possible.

JP 2006 305452 A relates to a filtration apparatus that uses a hollow fiber membrane. River water, lake water, groundwater or sea water is used. Filtration in hollow fiber membranes is effected from the outside to the inside, so that the purified water rises in the hollow fibers, for example, wherein the removed suspended materials are not able to penetrate the hollow fiber walls. Air bubbles are used in order to remove suspended particles adhering to the hollow fiber membrane, i.e., air bubbles hit the suspended particles adhering to the side surface of the hollow fiber membrane while the hollow fiber membrane vibrates. In this way, the suspended substance is peeled off the hollow fiber membrane. For detecting a rupture of the hollow fiber membrane, air bubbles are also added, so that air from the broken part enters the interior of the hollow fiber membrane. The bubbles are visible from the outside through a transparent viewing cap. Furthermore, it is described that the material with a minor crack can either be repaired or thrown away and replaced. A filtration system designed for contaminated water cannot be used for virus filtration. There is no step-by-step filling of the filtration apparatus, so a reliable integrity test cannot be effected. In addition, the volume flow of the gas through the filtration apparatus is not measured, so that the recognition of even the finest defects is not possible. In addition, the hollow fibers can be repaired or partially replaced here. Repair of a virus filtration module is not possible due to the delicate hollow fibers with tiny openings. Therefore, it can be assumed that the separation targets a completely different order of magnitude of the materials to be separated.

JP 3 557712 B2 and JP H07 136476 A disclose a method for detecting a defect in a hollow fiber membrane module by passing a gas through the module. If there is a defect in the hollow fiber membrane, fine particles, such as dust, are present in the gas passing through the hollow fiber membrane module. A laser beam is used for detecting the fine particles in the gas wherein the laser beam is scattered if the laser beam hits the fine particles, which can be captured by a CCD camera. According to JP 3 557712 B2, the diameter of the hollow fiber membrane module appears to be 5 mm or less, preferably 1 mm. This probably refers to the hollow fiber membranes themselves. Furthermore, a defect should be able to be repaired. There is no step-by-step filling of the hollow fiber membrane module with liquid, so a reliable integrity test cannot be effected. In addition, the volume flow of the gas through the hollow fiber membrane module is not measured, so that the recognition of even the finest defects is not possible. In addition, the hollow fibers can be repaired here. Repair of a virus filtration module is not possible due to the delicate hollow fibers with tiny openings. Therefore, it can be assumed that the separation targets a completely different order of magnitude of the materials to be separated. Virus filtration cannot be carried out with the hollow fiber membrane module.

Therefore, the present invention is based on the object of avoiding the disadvantages of the prior art and providing a test procedure for the integrity of virus filtration modules in the form of hollow fiber filter modules, which makes a reliable test procedure possible, the test results of which are reproducible, can detect even the smallest defects and minimizes the risk of damage to the hollow fibers during testing. An apparatus for partially or fully automated integrity tests is to also be provided.

### SUMMARY DESCRIPTION OF THE INVENTION

The above-described object is therefore achieved according to the invention by a method for testing the integrity of one or more virus filtration modules, comprising the steps of:
a) installing one or more virus filtration modules in a filtration apparatus, wherein each virus filtration module is in the form of a hollow fiber filter module, in each case comprising hollow fibers having an inner region and hollow fiber interstices, a feed side, a retentate side and a filtrate side, and in each case having a line on the feed side, a line on the retentate side and a line on the filtrate side;
b) filling the virus filtration module(s) and the connected lines with liquid at a constant pressure of at most 98±5 kPa with the following steps:
b1) filling all lines connected to the virus filtration module(s) with liquid, but without filling the virus filtration module(s);
b2) filling the inner region of the hollow fibers of the virus filtration module(s) with liquid, wherein the line on the feed side and the line on the retentate side are opened and the line on the filtrate side is closed and
b3) filling the interstices between the hollow fibers of the virus filtration module(s) with liquid, wherein in each case either the line on the feed side and the line on the filtrate side are opened and the line on the retentate side is closed, or the line on the retentate side and the line on the filtrate side are opened and the line on the feed side is closed; wherein in steps b2) and b3) the liquid is fed to the virus filtration module(s) from the line on the feed side or from the line on the retentate side;
c) stopping the feed of liquid as soon as the virus filtration module(s) and the lines are filled with liquid, wherein the pressure is maintained, and closing all lines;
d) feeding air to the virus filtration module(s) with step d1) or d2):
d1) opening in each case the feed-side line and the filtrate-side line of the virus filtration module(s) and leaving the retentate-side line closed, wherein the pressure is maintained, in order to feed air at a constant pressure at the same level as the liquid pressure from the feed-side line into the virus filtration module(s);
or
d2) opening in each case the retentate-side line and the filtrate-side line of the virus filtration module(s) and leaving the feed-side line closed, wherein the pressure is maintained, in order to feed air at a constant pressure at the same level as the liquid pressure from the retentate-side line into the virus filtration module(s);
wherein during step d1) or d2) the volume flow of air through the one or more virus filtration modules is measured; and
e) automatically detecting a chain of air bubbles exiting in the line on the filtrate side of the virus filtration module(s).

The disclosure also relates to a filtration apparatus for carrying out the method according to the invention, comprising
one or more virus filtration modules in the form of hollow fiber filter modules, in each case comprising hollow fibers having an inner region and hollow fiber interstices, in each case having a feed side, a retentate side and a filtrate side;
devices for attaching and fastening one or more virus filtration modules;
in each case, one line on the feed side, one line on the retentate side and one line on the filtrate side of each virus filtration module, for feeding liquid and/or air at a constant pressure of at most 98±5 kPa to the virus filtration module(s);
opening and closing devices for the respective lines;
a connector for feeding liquid at a constant pressure of at most 98±5 kPa having the lines to the virus filtration module(s);
a connector for feeding air of a constant pressure at the same level as the liquid pressure having the lines to the virus filtration module(s);
a detection apparatus for automatically detecting a chain of air bubbles in the line on the filtrate side of the virus filtration module(s) and
a measuring apparatus for measuring the volume flow of air through the virus filtration module (if only one virus filtration module is present) or through the virus filtration modules (if a plurality of virus filtration modules are present simultaneously).

The present disclosure thus makes possible, for the first time, a partially automated or fully automated testing of the integrity of virus filtration modules in the form of a visual leak test procedure, wherein the combined determination of the occurring chain of air bubbles and volume flow of air through each virus filtration module makes it possible to detect even the smallest defects.

The invention further relates to a method for cultivating prokaryotic or eukaryotic cells in liquid cell culture in a bioreactor to produce a product, in particular a protein, and carrying out virus filtration of the product obtained using one or more virus filtration modules, wherein the method for testing the integrity of the one or more virus filtration modules according to the present invention is carried out prior to and/or after virus filtration, in particular prior to and after virus filtration.

The invention also relates to a method for producing a recombinant protein, wherein the method comprises the following steps:
step a) cultivating prokaryotic or eukaryotic cells expressing a recombinant protein in cell culture in a bioreactor;
step b) harvesting the recombinant protein;
step c) purifying the recombinant protein;
step d) removing any viruses that may be present from the obtained recombinant protein in a virus filtration using one or more virus filtration modules, wherein the method for testing the integrity of the one or more virus filtration modules according to the present invention is carried out prior to and/or after virus filtration, in particular prior to and after virus filtration.

### BRIEF DESCRIPTION OF THE DRAWINGS

The embodiments of the present invention will be described by way of example with reference to the accompanying figures, which are drawn schematically and not to scale, so that no assumption of overly precise geometric values in relation to the original size can be made.

The figures of the present disclosure form part of the description and constitute a portion thereof, wherein they also illustrate embodiments of the invention without being restricted to the specific embodiments described. The drawings, together with the summary and detailed description, serve to explain the principles of the present disclosure. Where possible, the same features are provided with the same reference signs in the figures. FIG. 1 to 9 show schematic illustrations of exemplary embodiments of the present disclosure. In the figures in detail:

FIG. 1 is a schematic, simplified view of the interior of a virus filtration module in the form of a hollow fiber filter module;

FIG. 2 is a schematic, enlarged sectional view of a hollow fiber of the hollow fiber filter module of FIG. 1;

FIG. 3a is a schematic, enlarged front view of a virus filtration module in the form of a hollow fiber filter module with connected lines;

FIG. 3b is a schematic enlarged sectional view of an intact hollow fiber of the hollow fiber filter module of FIG. 1;

FIG. 3c is a schematic, enlarged sectional view of a non-intact hollow fiber of the hollow fiber filter module of FIG. 1;

FIG. 4 is a schematic, simplified view of a virus filtration module in the form of a hollow fiber filter module with connected lines on the feed side, retentate side and filtrate side according to an embodiment of the method according to the invention;

FIG. 5 is a schematic, simplified view of a virus filtration module in the form of a hollow fiber filter module that occupies an intermediate position between vertical and horizontal positions;

FIG. 6a is a schematic, simplified view of an embodiment according to the invention, wherein the relative position of the virus filtration module and the air filter to one another is shown by way of example;

FIG. 6b is a schematic, simplified view of a further embodiment according to the invention, wherein the relative position of the virus filtration module and the air filter to one another is shown by way of example;

FIG. 7 shows an exemplary setup for measuring the chain of air bubbles by optical means based on a scattered light sensor in a simplified form in a partial sectional view;

FIG. 8 is a schematic view of a section of a filtration apparatus according to an embodiment of the present invention, according to which a plurality of virus filtration modules can be tested simultaneously using the method according to the invention and

FIG. 9 is a schematic view of a section of a filtration apparatus according to a further embodiment of the present invention, according to which a plurality of virus filtration modules can be tested simultaneously using the method according to the invention.

### DETAILED DESCRIPTION OF THE INVENTION

### Definition of General Terms

Terms not specifically defined herein are to be given the meaning that a person skilled in the art would give them in light of the disclosure and context.

The term “integrity” in connection with a filter means detecting whether the filter is functioning properly or is impaired in its function. Therefore, an intact filter fulfills its filtering function in the desired manner. A “filter integrity test” serves to prove that a filter is or was (fully) functional and has functioned and is continuing to function without disruption, i.e., is/was undamaged and does/did not allow any undesirable components to pass through. The integrity test modified according to the invention is based on the integrity test with a manual test procedure according to the manufacturer Asahi Kasei's instructions for handling and testing in the general Planova virus filter testing procedure (“TAS 33088, Integrity Test Procedure Planova™ 15N, 20N, 35N and 75N Filters, Visual Leakage Test (VLT),” version 1.1 dated 25.02.2019), but is generally applicable to any type of virus filtration module.

The term “virus filtration module” represents a filter or filter unit that is designed and constructed to separate viruses from liquids by filtration. In the present disclosure, these are hollow fiber filter modules. The terms “virus filtration module” and “hollow fiber filter module” are used interchangeably in the present disclosure and are understood herein to be synonymous. The terms “virus filtration module” and “hollow fiber filter module” are abbreviated here and simply referred to as filter module or filter. According to the invention, virus filtration modules are used which are only approved up to a pressure of 98±5 kPa. There are numerous suitable virus filtration modules available on the market. The virus filtration modules used according to the invention are therefore also used for virus filtration at a maximum pressure of up to 98±5 kPa. According to the invention, virus filtration is therefore carried out, for example, at pressures in the range of 82 to 85 kPa or even 78 to 85 kPa.

The term “filter module” or “filtration module” is used synonymously with the “virus filtration module,” wherein the term “module” is understood to mean that, depending on the embodiment, the filter unit can also be supplemented by further filter units or modules, which can then be connected together and together assume the filter function as in a modular system.

A “filtration apparatus” is any type of apparatus that makes filtration with a virus filtration module possible. For the integrity test to be carried out, this comprises, for example, feed and discharge lines to the virus filtration module, means for closing and opening the feed and discharge lines, means for providing a liquid to be filtered, means for receiving a filtered liquid along with means for providing air. For a virus filtration to be carried out after the integrity test, this may, for example, additionally comprise: means for providing a product to be filtered, comprising liquid, along with means for receiving a product to be filtered, comprising liquid. The feed and discharge lines to the virus filtration module used for the integrity test can also advantageously be used for virus filtration.

“Hollow fiber filter modules” represent a technology known in the prior art in which hollow fibers with partially permeable walls act as filter membranes, which are combined in the form of fiber bundles to form filter modules. The hollow fibers have an inner region, which is also referred to as the hollow fiber inner side. There are also interstices between the hollow fibers in a hollow fiber filter module.

The expression “line on the filtrate side” means that this is understood to be the line that is connected to the filtrate side of the virus filtration module. Similarly, “feed-side line” means the line that is connected on the feed side of the virus filtration module. The “retentate side line” means the line that is connected to the retentate side of the virus filtration module.

The liquid used for the integrity test comprises or consists of water. For example, water can be present as the main component. For example, the liquid contains water in an amount of at least 90 wt. %, or at least 95 wt. %, or at least 98 wt. %, or at least 99 wt. %, or at least 99.5 wt. %.

The term “water” refers to any type of water that can be used. In particular, purified water is used, but in some embodiments tap water can also be used. The selection of water type depends on the intended use. Purified water as used in this disclosure is water that has been subjected to a purification process such as distillation, reverse osmosis, carbon filtration, capacitive or electrode ionization, micro-or ultrafiltration, ultraviolet oxidation or the like in order to remove impurities and make it suitable for use. Combinations of these methods can also be used in order to obtain water of high purity, such as ultrapure water, wherein its trace contaminants are measured in parts per billion (ppb) or parts per trillion (ppt). For example, distilled water, bidistilled water or deionized water can be used. In one embodiment, the water used in the method according to the invention is ultrapure water, e.g., Type 1 ultrapure water according to ASTM D1193 or ISO 3696. According to another embodiment, the water used can be sterile water that is suitable for administration to a subject (human or animal), such as water for injection (WFI).

The term “process air” is understood to mean pressurized air that is used directly as a process medium in the method. Pressurized air can fulfill certain quality and purity criteria, is usually dry, germ-free and can even be sterile. According to the invention, outside air taken from the environment is used as process air. It is compressed air, i.e., pressurized air, wherein, however, a pressure of at most 98±5 kPa is used. The atmospheric pressure is 101.3 kPa.

The term “transparent” refers to materials that allow the majority of light to pass through, wherein a small part of the light is usually absorbed and scattered. In contrast, materials with delimited light transmission are not transparent and are also referred to as opaque. Therefore, transparency is the ability of matter to allow light waves to pass through (transmission). Transparent materials are characterized by high transmittance. Here, the term “transparent” means that the material used has a high light transmission, which means that there is a light transmission that is at least 75% or at least 80% or at least 85% or at least 90% or at least 95% of the light of the wavelength used and at the thickness of the material used. Transparent materials used here are those that can be used under the selected method conditions, i.e., they can withstand a pressure of up to 98±5 kPa and are not attacked by the liquid used, in particular water.

### Embodiments of the Invention

The individual steps of the integrity test of the present disclosure with its different embodiments will be explained in detail below, wherein the method will initially be described with reference to an embodiment with a virus filtration module: Before the method for testing the integrity of a virus filtration module according to steps a) to e) is carried out, it may be expedient if sterilization, in particular hot steam sterilization, of the lines is carried out. This is usually carried out without a virus filtration module, wherein a fitting piece is inserted into the filtration apparatus for closing the open section instead of the virus filtration module. Sterilization can, for example, be effected at 100° C. or more, in particular in the range of 100-130° C. This is useful if the integrity test is to be followed by virus filtration.

According to one embodiment of the invention, it is useful if, after sterilization of the lines and prior to carrying out step a), the lines are placed under an overpressure of, for example, approximately 100 kPa, in order to prevent the introduction of contamination after sterilization. This is then followed by a pressure reduction, for example to a pressure P≤10 kPa, in all lines, wherein pressure equalization is effected in the entire system. This serves to protect the virus filtration module from damage.

In step a) of the present method for testing the integrity of a virus filtration module, a virus filtration module is initially provided and installed in a filtration apparatus. The filtration apparatus is an apparatus in which the integrity test of the virus filtration module is to be carried out. Here, the lines of the virus filtration module are connected to suitable liquid or air connections in a functional manner. The filtration apparatus is also designed in particular so that virus filtration can be carried out using the virus filtration module.

Here, the virus filtration module represents a hollow fiber filter module that is to be used in a method for removing viruses from a liquid. The hollow fiber filter module comprises hollow fibers having an inner region, also referred to as the hollow fiber inner side, and hollow fiber interstices. The virus filtration module in the form of a hollow fiber filter module is not further restricted; any hollow fiber filter module known to a person skilled in the art that can be used to remove viruses can be used, provided that it is approved for virus filtration at a pressure of up to 98±5 kPa. By way of example, the hollow fiber filter modules Planova™ 15N (hollow fiber diameter 15 nm), 20N, 35N, or 75N from Asahi Kasei may be mentioned, which are used for removing viruses. Virus filtration modules approved for higher pressures are not used according to the invention.

The viruses that can be removed with the virus filtration module are also not further restricted. Using known virus filtration modules, almost any type of virus can be removed from liquids. For this purpose, these include, for example, parvoviruses, which are among the smallest viruses found in nature. Even larger viruses, such as, e.g., retroviruses, can be removed using virus filtration modules.

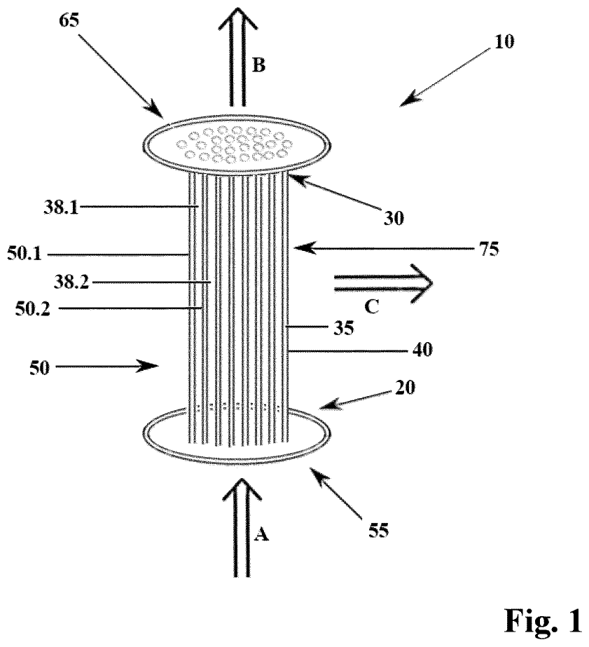

The general setup of such virus filtration modules in the form of hollow fiber filter modules has already been explained in detail in FIG. 1. They have a feed side, a retentate side and a filtrate side, in each case with lines connected thereto. The material of the lines is not particularly restricted as long as it does not adversely affect the carrying out of the integrity test. Therefore, materials suitable for the method conditions of the integrity test are used, i.e., a pressure of up to 98±5 kPa and the use of liquid, including water, as the liquid medium. For example, plastics or metal, in particular stainless steel, are used.

The commercially available hollow fiber filter modules usually have two inlets and two outlets: For the two inlets, which are defined by the two ends of the hollow fibers, one end is selected as the feed for the liquid to the hollow fiber filter (the so-called feed side) and one end is selected as the discharge for the liquid from the hollow fiber filter (the so-called retentate side). Therefore, one of the two inlets is selected as outlet. Both inlets are completely equal, so that the selection is effected only pro forma. The two outlets of the hollow fiber filter modules are analogous to the inlets, which can be flexibly selected. Both outlets are also completely equal, so that the selection is effected only pro forma. The selection of inlet and outlet does not change the filtration or the test result. The actual filtration is effected through the wall of the hollow fibers, so that the filtrate is obtained through the wall of the hollow fibers (the so-called filtrate side) via two outlets. For carrying out the integrity test procedure according to the disclosure, only one inlet and one outlet are required at a time, so that further inlets and further outlets are closed during the test.

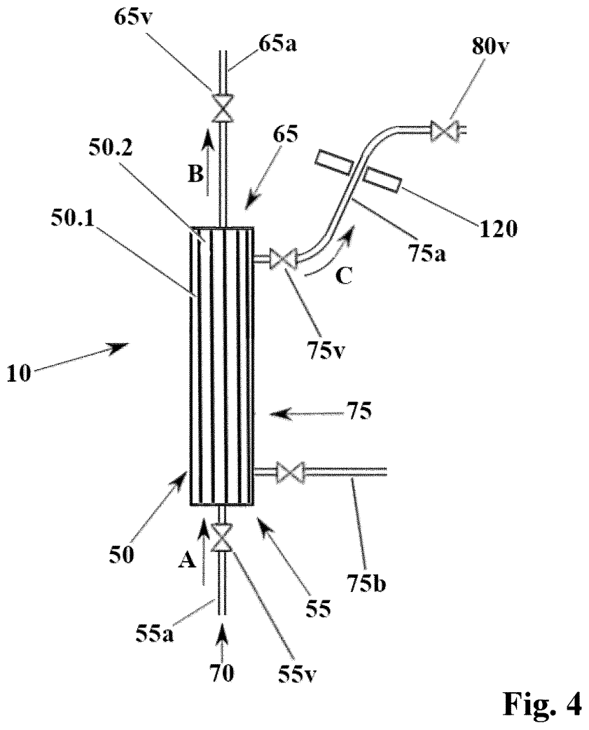

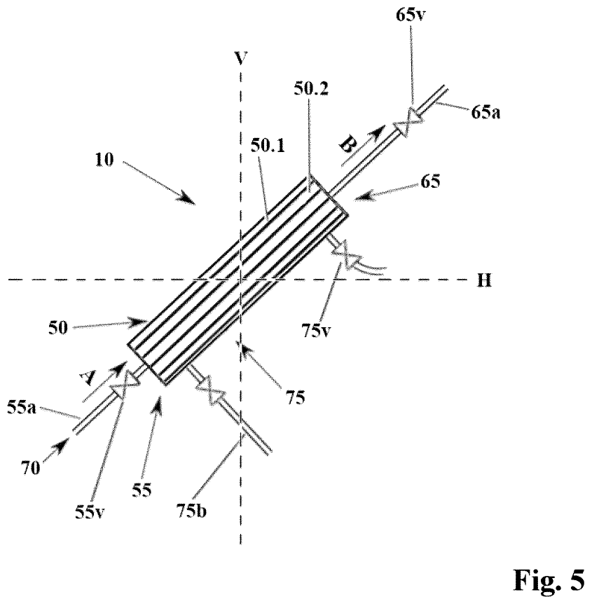

The arrangement of the virus filtration module in step a), i.e., the alignment of the hollow fibers when installing the virus filtration module in a prepared filtration apparatus in which the integrity test is to be effected, can be completely arbitrary. There is actually no preferred orientation. The virus filtration module can, for example, be arranged with the hollow fibers aligned horizontally or vertically or in an intermediate position between horizontal and vertical directions. According to one embodiment, it may be advantageous if the hollow fibers in the virus filtration module run in a vertical direction or in an intermediate position between horizontal and vertical directions. The term “in an intermediate position between horizontal and vertical directions” means that a virus filtration module is not positioned upright, as shown in FIG. 4 by way of example; rather, it is inclined by an arbitrary angle, but is not yet positioned in the horizontal direction (quasi lying down) (see FIG. 5 for an exemplary intermediate position of a virus filtration module). According to one embodiment, the hollow fibers are in particular parallel to one another during the integrity test. An upright (vertical) position of the virus filtration module, as schematically shown in FIG. 1, was selected for reasons of simplified representation and better describability.

In step b) of the method, the virus filtration module and the connected lines are filled with liquid, including water. For this purpose, a liquid pressure of at most 98±5 kPa is used. The pressure is in a range that can be easily used with virus filtration modules and does not adversely affect or even damage them. Significantly lower pressures are also possible, such as the following lower limits: 10±0.5 kPa or 20±1 kPa or 30±1.5 kPa or 40±2 kPa or 50±2.5 kPa or 60±3 kPa or 70±3.5 or 80±4.0 or 90±4.5, wherein the upper limit is 98±5 kPa. The higher the pressure is set, the more easily the chain of air bubbles can be detected. If a liquid pressure within the above range is selected, it will be maintained throughout the integrity test. The integrity test can also be carried out at a low pressure of 10±0.5 kPa or 20±1 kPa, since a chain of air bubbles can also be detected in this case. According to one embodiment, the same or a lower pressure as in the integrity test of the invention is selected in a virus filtration carried out with the tested virus filtration module(s).

Filling with liquid is effected in 3 sub-steps, wherein the liquid has a constant pressure of at most 98±5 kPa, which—as already explained—is selected to be the same in all 3 sub-steps:
In step b1), the filling of all lines connected to the virus filtration module is effected with liquid, but without filling the virus filtration module.
In step b2), the inner region or the hollow fiber inner side of the hollow fibers of the virus filtration module is filled with liquid, wherein the line on the feed side and the line on the retentate side are opened and the line on the filtrate side is closed.
In step b3), the interstices between the hollow fibers of the virus filtration module are filled with liquid, wherein either the line on the feed side and the line on the filtrate side are opened and the line on the retentate side is closed, or the line on the retentate side and the line on the filtrate side are opened and the line on the feed side is closed.

In steps b2) and b3), the liquid can be fed to the virus filtration module from the line on the feed side or from the line on the retentate side. If the virus filtration module is installed vertically in the filtration apparatus, the liquid feed from the feed side means that the liquid is fed to the virus filtration module from below. Feeding from the retentate side means that the liquid is fed into the virus filtration module from above. For practical reasons, it is advantageous if the liquid is fed from the same side in step b2) and in step b3), but this need not be the case.

As already mentioned, the orientation of the virus filtration module is completely arbitrary. The vertical alignment of the hollow fibers and thus also of the virus filtration module is only mentioned in order to be able to better illustrate the processes. Here, the expression “feed from below” or “from above” does not mean that a virus filtration module must in every case assume an exactly vertical position. Rather, the virus filtration module can also assume any intermediate position other than the vertical alignment, which lies between a vertical and a horizontal alignment (see FIG. 5). Even if the virus filtration module is inclined at an angle away from the vertical direction, feeding can be effected from below or above.

The liquid used is usually water or a buffer solution. There are no particular restrictions on the water used. Depending on the intended use of the filter module, the type of water and its degree of purity can be selected. However, it is understood that this should not contain any components that could in any way impair the virus filtration module or the integrity test. The water is, for example, distilled water, bidistilled or deionized water or Type 1 ultrapure water according to ASTM D1193 or ISO 3696, in particular sterile water suitable for administration to a human or animal, such as, e.g., water for injection (WFI). This depends on the intended use, in particular from which liquid product the viruses are to be removed later.

According to one embodiment, a buffer solution is used which typically contains 95 to 99.5 wt. % water, such as WFI water (water for injection). The buffer solution contains, in addition to water, a component such as acetate/acetic acid, and can also contain salts such as NaCl, solvents such as ethanol, and additives such as sugars, for example sucrose, and one or more amino acids, e.g., histidine. The advantage of such buffer solutions is that they can have better compatibility with a product to be filtered and, for example, increase the stability of a product used. The presence of salts also has the advantage that the electrical conductivity of the liquid can be measured. This plays a role if WFI water, which has practically no electrical conductivity, is used as the water. Therefore, the composition of the buffer solution is selected specifically for the product.

The filling of the virus filtration module having liquid according to steps b2) and b3) can be effected from the feed side or from the retentate side. If the virus filtration module were in a vertical position, the feed side would be at the bottom and the retentate side at the top. However, the virus filtration module can assume any position during filling, i.e., during the entire integrity test and, if applicable, also during the virus filtration carried out. However, it is expedient to maintain the orientation or position of the virus filtration module(s) once set during the method, since otherwise the virus filtration module(s) would have to be removed from a system and reinstalled, which is not useful in terms of method technology.

According to one embodiment, the liquid is fed from the feed side, for example from below, into the inner region and the interstices of the virus filtration module. Adding the liquid from below can be advantageous, since, as a rule, this is simpler in terms of method technology. A further advantage of feeding the liquid from below is that any air present in the filter module is flushed out of the filter upwards, so that no air can remain in the filter. At the same time, complete venting of the filter module can be achieved.

In step b1), the filling level of the lines can be checked so that there are no more air bubbles in the outflowing liquid. This can be effected visually by an employee or with a sensor.

In steps b2) and b3), the filling level of the virus filtration module with liquid can be monitored by measuring the pressure prior to and after the filter module and determining the differential pressure. This is effected, for example, by measuring the pressure in the line through which the liquid is fed to the virus filtration module and in the line through which the liquid is discharged from the virus filtration module and calculating the difference between the two pressures. This can be carried out in both step b2) and step b3). The lower the differential pressure, the higher the filling level. At a pressure difference of maximum 98±5 kPa, it can be assumed that the virus filtration module is filled according to step b2) or b3).

Additionally or alternatively, the amount of liquid fed can be set in relation to the dead volume of lines and virus filtration module, wherein the following applies: the amount X of liquid fed≥ dead volume. In this case, the virus filtration module would then be filled. The dead volume refers to all volume portions of the system under consideration that are to be filled with liquid. The dead volume for step b2) and step b3) can be easily determined by a person skilled in the art. The dead volume can be obtained from the manufacturer's specifications or can be requested from the manufacturer. For example, a 4 m² filter module has a dead volume of 1,900 mL according to the manufacturer's specifications. When testing a plurality of filter modules, their dead volumes are cumulative.

The virus filtration module is completely filled with liquid so that—as far as possible—no empty space remains. In other words, all hollow fiber interior spaces (step b2)) along with all interstices (step b3)) between the hollow fibers in the filter module are filled with liquid. In addition, the lines connected to the virus filtration module are completely filled with liquid. This is effected by adding the liquid under pressure, wherein the pressure is set within the specified range.

Prior to the entry of the liquid into the virus filtration module according to steps b2) and b3), an inlet filter can be provided in the feed line which filters out possible impurities and contamination from the liquid and prevents them from entering the virus filtration module. Therefore, this inlet filter can be provided in the line on the feed side and/or in the line on the retentate side. In the same way, it may be expedient to install an outlet filter in the discharge line at the outlet from the virus filtration module. This also prevents possible impurities and contamination from being present in the liquid and thus impairing subsequent detection.

As soon as the virus filtration module and the lines are completely filled with liquid, the feed of liquid is stopped in step c), wherein, however, the applied pressure is maintained. For this purpose, all lines are closed, so that a closed system is created that is completely filled with liquid and remains under the applied pressure of at most 98±5 kPa.

The filling of the virus filtration module(s) and the connected lines with liquid at a constant pressure of at most 98±5 kPa is carried out in 3 sub-steps. As a result, the virus filtration module(s) is/are completely filled with liquid. Tests have shown that filling a virus filtration module in a single step is not possible, since the liquid seeks the path of least resistance and then does not fill all the empty spaces in the virus filtration module. A reliable integrity test cannot then be carried out. Carrying out the filling process in three sub-steps therefore means not only a particularly gentle, but also a controlled procedure. The division of the filling process into three sub-steps also allows for a triple check, in which in each step a sub-portion can be checked and verified to ensure that the filling has been completed. This can be precisely determined, for example, based on the amount of liquid fed in relation to the dead volume (e.g., based on the manufacturer's specifications), as already explained. The orientation of the virus filtration module generally plays no role during filling, since the step-by-step filling is effected completely independent of the orientation. The virus filtration module can be in a vertical or horizontal position or inclined at any angle other than that, in particular in an intermediate position between a vertical or horizontal position. If an orientation of a virus filtration module is given by way of example, this is only for better understanding and a simplified description.

According to an alternative embodiment, an optional intermediate step can be carried out after step c), in which the existing liquid is completely emptied again only from the inner region of the hollow fibers. If the virus filtration module in the filtration apparatus is arranged vertically or in an intermediate position between the vertical and horizontal directions, this can be effected, for example, by opening the line on the feed side, as a result of which the liquid drains downward. This is effected until the inner region of the hollow fibers and the connected line have been completely emptied. This can be monitored by ensuring that no more liquid exits the outlet of the filtration unit. This can be effected by visual inspection or via a sensor.

A procedure according to which the filling of the inner region of the hollow fibers according to step b2) is omitted and only the interstices are filled according to step b3) is not possible for physical reasons. Therefore, the entire virus filtration module must first be filled before the liquid can be drained from the inner region.

This optional intermediate step is not necessary in every case. This intermediate step has the advantage that the internal emptying of the virus filtration module represents a particularly gentle procedure for the subsequent integrity test. If the inner region or the inner side of the hollow fibers is filled with pressurized liquid and then pressurized air is also fed for the integrity test, this could result in the hollow fibers swelling, as a result of which the stress on the filter module could further increase and the tendency for damage could rise.

Prior to carrying out step d), a leak test of the complete system, including the virus filtration module, can be carried out in order to rule out any leaks in the system, e.g., due to an improperly installed seal. This serves to ensure that no air escapes from the system and could thus falsify the subsequent integrity test and its evaluation. However, since according to the invention the (total) volume flow of air through the filtration system and thus through the virus filtration module (if only one virus filtration module is tested) or through the virus filtration modules (if a plurality of virus filtration modules are tested simultaneously) is measured during the method, a leak test is no longer necessary, since the size of the volume flow immediately shows if the system contains leaks. For the sake of completeness, a possible embodiment of a leak test will nevertheless be described here.

The leak test is also referred to as the pressure hold test. With this, air is fed to the virus filtration module via the retentate side or via the feed side, a pressure below 98±5 kPa, since the so-called pre-stabilization pressure is selected, for example in the range of 60-70 kPa, and after a stabilization period of at least 180 s, the pressure drop in the system is measured.

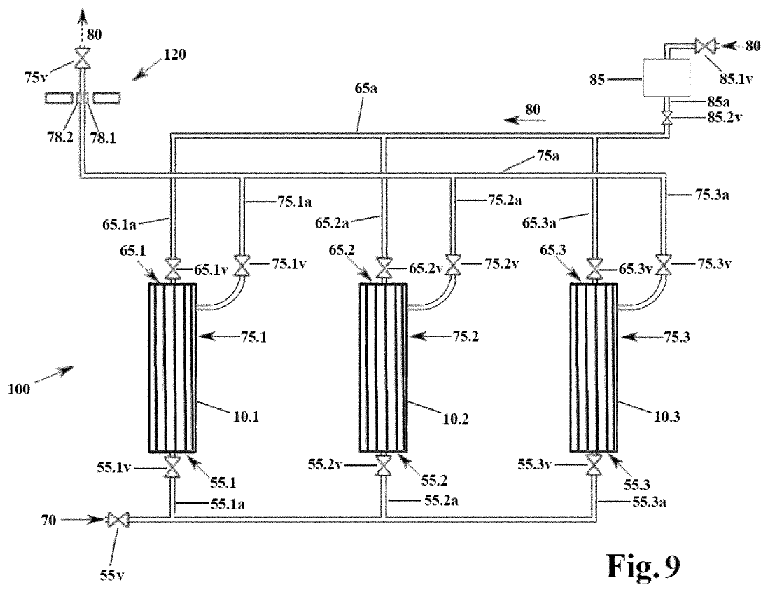

In the leak test, in detail, the lines filled with liquid at the virus filtration module are opened (e.g., the lines 55a and 75a or 65a and 75a in FIG. 4). A further outlet on the filter module is closed (e.g., in FIG. 4: outlet at 75b). Inlets and outlets not required of the system are closed (e.g., inlet and outlet for water 70 and air 80 in FIG. 9). Air, such as process air, is then fed to the virus filtration module. This can be effected either from the retentate side (in FIG. 4: at 65a) or from the feed side (in FIG. 4: at 55a) of the virus filtration module. If the virus filtration module is installed in the filtration apparatus in a vertical position as shown in FIG. 4 or in an intermediate position between vertical and horizontal position, the air is fed via the retentate side from above or via the feed side from below. Other orientations are possible. A step-by-step pressure buildup was effected by means of a controlled pressure increase (e.g., a fixed ramp) of the fed air until a test pressure, also referred to as the pre-stabilization pressure, was reached. The air pressure in the system can, for example, be increased by a time ramp (kPa/min) starting at 0 kPa to a value in the range of 60 to 70 kPa. The pre-stabilization pressure is selected to be lower than the pressure during the integrity test (maximum 98±5 kPa). It may be expedient to observe a stabilization period after reaching the pre-stabilization pressure, during which the air can be dispersed within the system before the lead test is evaluated. The stability time is at least 180 s. The leak test is considered passed if the pressure drop after the stabilization period is ≤10 kPa, in particular ≤5 kPa. If the pressure drop is ≤10 kPa, it must be assumed that there is no leak in the filtration system. If the pressure drop is >10 kPa, it must be assumed that there is a leak in the system. In this case, the leak in the filtration system is identified and eliminated.

After step c) or after the optional intermediate step described above or after the optional leak test described, air is fed to the virus filtration module with step d1) or d2). The two method variants result from the fact that the air is fed into the virus filtration module from either side. If the virus filtration module is installed with vertically arranged hollow fibers or in an intermediate position between vertical and horizontal, the air is fed from above or below. If the virus filtration module is in a different orientation, the air is fed either from one side or the other. However, it is difficult to describe the complex relationships if one side is to be distinguished from the other. Therefore, to simplify the following explanations, the vertical alignment of the virus filtration module is assumed in order to be able to simply refer to “bottom” or “top,” even if this vertical alignment does not have to be exact when carrying out the method. The virus filtration module could also be installed in the filtration apparatus in a different alignment.

In detail, according to the first method variant, in step d1), the line on the feed side (in the described embodiment, by way of example, at the lower end of the filter module) and a line on the filtrate side of the filter module (on the wall side of the filter module) are opened and the line on the retentate side (at the upper end of the filter module) and, if applicable, another line not used for the test on the filtrate side of the filter module (on the wall side of the filter) are in each case left closed in order to force air into the virus filtration module (in the described embodiment, for example, from below) via the line on the feed side at a constant pressure at the same level as the liquid pressure, i.e., a pressure of at most 98±5 kPa. Opening the lines does not mean that the lines are opened to the outside. Rather, the pressure of at most 98±5 kPa is fully maintained in the virus filtration module and the connected lines. The only purpose of opening is to allow air under pressure to be fed. It is known to a person skilled in the art how to feed air to a pressurized system at the same pressure.

In the alternative, second method variant, in step d2), the line on the retentate side (in the described embodiment, by way of example, at the upper end of the filter module) and a line on the filtrate side of the filter module (on the wall side of the filter module) are opened and the line on the feed side (in the described embodiment, by way of example, at the lower end of the filter module) and, if applicable, a further line not used for the test on the filtrate side of the filter module (on the wall side of the filter) are left closed in order to force air into the virus filtration module (in the described embodiment, by way of example, from above) via the line on the retentate side at a constant pressure at the same level as the liquid pressure, i.e., a pressure of at most 98±5 kPa. Opening the lines does not mean that the lines are opened to the outside. Rather, the pressure of at most 98±5 kPa is fully maintained in the virus filtration module and the connected lines. The only purpose of opening is to allow air under pressure to be fed. It is known to a person skilled in the art how to feed air to a pressurized system at the same pressure.

The opening and closing of the lines can be effected by means of suitable devices, such as so-called shut-off devices. For this purpose, e.g., a ball valve may be used, which only has the states “open” and “closed.” Also possible are valves of all kinds, such as control valves that allow continuous actuation between 0 and 100%, automatic valves that only have open and closed positions, or manually operated hand valves. Other options are also known from prior art to a person skilled in the art.

The selected pressure for the liquid and thus also the fed air is in a range that can be easily used with virus filtration modules and does not adversely affect or even damage them.

It goes without saying that pressure monitoring can also be carried out during the test procedure by means of pressure measuring and control devices. This is self-evident to a person skilled in the art and is not described explicitly.

The fed air is therefore forced from a filter inlet of the virus filtration module through the hollow fibers contained in the module to the filter outlet. In this case, the filter inlet is the side of the filter where the air is introduced. In the first method alternative d1), the filter inlet for the air is therefore the feed side of the filter (lower end of the hollow fibers), in the second method alternative d2), the filter inlet for the air is the retentate side of the filter (e.g., upper end of the hollow fibers). The filter outlet is always the filtrate side, where the air can or cannot exit, depending on whether or not the filter is intact.

According to one embodiment, if the virus filtration module is installed vertically or in an intermediate position between vertical and horizontal direction, in particular the second method variant is used, in which the air is fed from above (upper end or retentate side of the virus filtration module). The second method alternative is advantageous because it has method-related advantages. It has proven to be more advantageous if air is introduced into the filter module from the highest point.

According to one embodiment of the invention, it may be expedient for method-related reasons if, in the explained leak test of the lines and the virus filtration module, the air is fed into the virus filtration module from the same side, e.g., the retentate side, as in the integrity test (step d1) or d2)).

The air fed under pressure serves to detect whether the hollow fibers in the virus filtration module are intact, i.e., they do not exhibit any damage, such as cracks, and therefore fulfill the desired filtering function. When the virus filtration module is functional, the air remains in the hollow fibers, since the air cannot penetrate the pores of the hollow fiber wall and can penetrate into the closed line at the filter outlet (retentate side or feed side of the filter module, depending on where the air was injected). If the virus filtration module is not functional, the air passes through damages in the hollow fiber wall and reaches the filtrate side of the filter module, where a chain of air bubbles can then occur and be detected in the connected line on the filtrate side.

The air used is not particularly restricted. However, it is understood that it should not contain any components that could in any way impair the virus filtration module or the integrity test. Process air is preferably used.

According to one embodiment, the air used in method steps d1) and d2) is in each case initially passed through an air filter before it can enter the virus filtration module in order to retain potential impurities and contamination from the air, so that they cannot enter the virus filtration module. It has been found that the test procedure is not impaired in any way by such an air filter.

According to one embodiment, the air filter is arranged above the virus filtration module, i.e., the air filter is located completely above the virus filtration module. If an air filter is provided and the virus filtration module is installed in a vertical position (with vertically running hollow fibers) or in an intermediate position between vertical and horizontal direction, it is particularly advantageous if the liquid is introduced from below (step b), 1st alternative) and the air is introduced from above (step d2)) into the virus filtration module, since this procedure makes it easier to prevent the air filter from being flooded with liquid and thus impaired in its function.

According to a further embodiment, the air-conducting line leading from the air filter can have a portion that comprises the highest point of the filtration apparatus. As a result, it is ensured that liquid cannot rise up to the air filter and penetrate it. In this case, it does not matter where the air filter is located; it can also be located below the virus filtration module. This does not have to be the highest point of the system; rather, it is sufficient if the line leading from the air filter has the highest point of the apparatus. These embodiments are explained in detail in the figures.

Even if the virus filtration module is completely intact, tests have found a minimal loss of injected air after passing through the virus filtration module. This small amount or leak flow can only be detected based on an air measuring device, where this can be a few ml/min, for example 3-6 mL/min. This leak flow of air does not lead to the formation of a chain of air bubbles on the filtrate side of the virus filtration module, which must be present for at least 20 s. In contrast, if a virus filtration module is not intact, it has been found that large amounts of air, for example in the range>150 mL/min, exit on the filtrate side, so that a clear distinction can easily be made between an intact and a non-intact virus filtration module.

In addition to checking the criterion of whether a continuous chain of air bubbles occurs, in particular over a duration of at least 20 seconds, a further parameter is checked during the integrity test, during step d1) or d2), in order to detect whether an intact or defective virus filtration module is present. This involves measuring the volume flow of air through each virus filtration module, in particular measuring the total volume flow through all virus filtration modules. Therefore, in steps d1) and d2), the (total) volume flow of air is additionally measured, which indicates how much volume of a medium (here: air) is transported per period of time through a specified cross section (here: the virus filtration module(s)). In particular, step e) is carried out while step d1) or step d2) is still being effected, so that in particular the volume flow of air through the virus filtration module(s) continues to be measured during step e).

According to one embodiment, the volume flow of air through the virus filtration module(s) can also be measured only in step e). This has no adverse effect on the test result and represents a variant of specifying the test period that only plays a role in terms of measurement technology. Therefore, the test period begins in step d) and ends after step e) or the test period begins in step e) (while step d) continues) and ends after step e). Therefore, the test period can start earlier or later during step d), which-depending on the management of the method-can be determined by the feed of air and the achievement of the selected pressure.

In order to determine whether an intact or defective virus filtration module is present, a comparison is undertaken of the volume flow of air of an intact virus filtration module with a defective virus filtration module. It has been found that an intact virus filtration module has a volume flow of injected air in the range of approximately 0.8 to 8.0 mL/min/virus filtration module, whereas a defective virus filtration module has a significantly higher volume flow. Therefore, a volume flow of air of 8 mL/min/virus filtration module is assumed for an intact virus filtration module. This represents a (maximum) expected value with which a comparison is effected in order to exclude diffusion effects. Therefore, the volume flow of 8 mL/min per virus filtration module corresponds to the maximum volume flow (Volₘₐₓ) permitted per virus filtration module. In particular, it is checked whether the maximum permitted volume flow Volₘₐₓ of the air is maintained throughout the entire test period.

According to the invention, the volume flow of air can be measured individually for each virus filtration module. In particular, however, a total volume flow is measured in a single measuring apparatus for all existing virus filtration modules together. In this connection, it should be noted that the volume flows, measured as total volume flow, add up when a plurality of virus filtration modules are tested in parallel: If an intact virus filtration module has a volume flow of air of approximately 8 mL/min per virus filtration module, seven virus filtration modules that are simultaneously subjected to the described integrity test have a total volume flow of air of maximum approximately 56 mL/min. In other words, in step d 1) or step d 2), a maximum total volume flow for air of at most 56 mL/min is measured if all seven virus filtration modules are intact. Here, the maximum volume flow of air s also abbreviated as Volₘₐₓ. According to the invention, the measured maximum volume flow Volₘₐₓ of air must not exceed 8 mL/min per virus filtration module.

A test was now carried out in which one hollow fiber of the hollow fiber filter module was intentionally destroyed and the others were left intact. The volume flow of air through the hollow fiber filter module was then measured. This was more than 150 mL/min for the virus filtration module. It can therefore be assumed that the measured volume flow of air of more than 150 mL/min indicates a defective virus filtration module. It has been found that by measuring the (total) volume flow of air, even the smallest defects, such as the finest cracks, in the virus filtration modules can be detected.

According to one embodiment, the volume flow of air can be measured at a position that is arranged, for example, before the introduction of air into the hollow fiber filter module (step d1) or d2)). This is because it is a closed system, so that the volume flow of air could be measured at any point in this closed system.

According to one embodiment, between step d) and step e), more precisely while step d) is still being carried out, but prior to step e) being carried out, a certain period of time can be provided, which is observed, during which the air can continue to be fed and can be further dispersed in the system. This is the so-called stabilization period, which can be up to 600 s. According to a further embodiment, this stabilization period can also be omitted.

Step e), which follows step d), is carried out so that the feed of air according to step d) is continued during step e), i.e., in step e), air is still fed, wherein, if applicable, after a stabilization period of up to 600 s, an automatic detection of any chain of air bubbles that may occur in the line on the filtrate side of the virus filtration module is carried out. Therefore, step e) is effected while step d) continues to be carried out. The detection of a chain of air bubbles occurs particularly in the line at the filter outlet on the filtrate side of the virus filtration module. Thus, a chain of air bubbles that occurs on the filtrate side of the filter module when the filter module is not functional is automatically captured.

The detection of a possible defect in the filter in the form of a chain of air bubbles is effected by optical means, for example. The measuring principle is based on the fact that a light beam that is passed through a liquid changes when air is present in the liquid. This is similar to the well-known measurement of the turbidity of a liquid caused by small suspended (undissolved) particles or small droplets that have a different refractive index than the surrounding medium. This difference leads to reflection, absorption and scattering, i.e., a change in the direction of the incident light. The intensity and spatial distribution of the scattered light depend on the wavelength of light used and the size of the particles or droplets or, in this case, the presence and size of the air bubbles.

The presence of air in the liquid is therefore determined by an optical measuring method, followed in particular by an evaluation of the optical measurement in an automatic form. In general, two measurement methods are distinguished: The weakening of the transmitted light radiation (so-called transmitted light) and the lateral scattering of the light radiation (so-called scattered light). In the present disclosure, scattered light measurement is used in particular. This represents a measurement technology that is already established on the market and is known from the prior art to a person skilled in the art. Scattered light measurement represents a highly sensitive and reliable optical method that is also known to be used for measuring very low turbidity. Scattered light measurement has proven to be a particularly suitable measurement method for the automatic detection of air bubbles in a liquid, including water. For automated detection by optical means, a counting apparatus for the chain of air bubbles can be used, such as an air bubble counter.

The wavelength of the measuring radiation is, for example, in the near infrared (NIR), in particular in the range from 730 to 970 nm. However, other wavelengths are also possible, such as a white light lamp (e.g., tungsten lamp) with wavelengths in the range between 400 and 600 nm.

During the measurement, for example, light in the near infrared range having a wavelength in the range of 730 to 970 nm is emitted by the sensor, passes through the line filled with liquid and is modified by the air present in the liquid. The light exiting the line can then be captured, for example, via diodes or photocells, and a measuring signal can be ascertained from this. Here, the measuring signal or measuring unit is the absorption, so that a specific absorption signal-more precisely, the change in the absorption signal caused by the presence of air-is detected. So-called scattered light sensors, which measure light scattering (side scattering), are used in particular to measure scattered light. A scattered light sensor is typically constructed from a light source and one or more detectors for detecting the light passing through the line. For example, commercially available scattered light sensors, in particular well-known turbidity sensors, can be used.

Therefore, the scattered light sensor is located particularly at the filter outlet on the filtrate side of the virus filtration module, namely on the line connected on the filtrate side. According to one embodiment, the scattered light sensor is arranged on a line running from bottom to top, so that the chain of air bubbles in the line can be completely detected as it rises.

For detection by optical means, the line on the filtrate side is selected so that it consists of or comprises transparent material. Therefore, the line as a whole can be made of transparent material. Alternatively, the line is constructed of transparent material only at the location of detection, so that optical detection is possible. In other words, the line is transparent at the positions where the light enters and exits the line.

The entire line at the location of detection does not have to be transparent; rather, only selected portions of the line can be transparent.

For example, the portion can represent a transparent tube that replaces part of the line, or there can be two opposite transparent windows attached in or on the line, or the entire line can be selected to be transparent.

The transparent portion can, for example, be a transparent tube that has a smaller diameter than the line and replaces part of the line. As a result, the line can be split on the filtrate side and plugged onto the transparent tube on both sides, wherein the transparent tube is arranged at the location of detection, i.e., where the scattered light sensor is located.

Alternatively, there can be two transparent windows, either integrated into the line wall or attached to the line in the beam path of the measuring light, on the outer side or inner side of the line, wherein one window is provided at the light entry into the line and one window at the light exit from the line. It is understood that the transparent portions, such as a tube or two windows, are integrated into or attached to the line in such a way that the line does not lose its tightness and remains tight. Corresponding seals can be used for this purpose. Other options than those described are also conceivable for a person skilled in the art.

Transparent materials are those that are suitable for carrying out the integrity test and tolerate the method conditions without being adversely affected. The method conditions that play a role here are a pressure of up to 98±5 kPa and resistance to the effects of the liquid used, including water. It can also be advantageous if the material can withstand high temperatures of up to 140° C.

For example, transparent plastics are used as transparent materials. These include, for example, polystyrene (PS), polymethyl methacrylate (PMMA), polycarbonate (PC), polyvinyl chloride (PVC), polyphenylene ether (PPO) or polyethylene (PE) or mixtures of these.

Glasses can also be used as transparent materials, in particular those that are stable against the attack of liquids, including water, and can withstand a pressure of up to 98±5 kPa. According to one embodiment, for example, a glass is used that has a hydrolytic resistance of class 1 according to ISO 720 or USP660. These include, for example, borosilicate glasses such as Duran®, Pyrex® or Fiolax®.

For example, sapphire, also known as sapphire glass, can also be used. Sapphire or sapphire glass is not glass, but high-purity, synthetic sapphire (single crystal), which is made from molten aluminum oxide (Al₂O₃). This material is particularly suitable, since it is transparent, suitable for the pressures used, is not attacked by the liquid, including water, and can withstand high temperatures, for example up to 140° C., without problems.

Since sapphire is very expensive, according to one embodiment, in particular only a part or parts of the line on the filtrate side are selected from sapphire. In particular, two sapphire glass windows are used at the location of measurement.

According to a further embodiment, plastics material that is not transparent is used for the line on the filtrate side, wherein two glass windows or two sapphire glass windows are built into the line at the location of measurement for the measurement by optical means.

According to a further embodiment, metal, in particular stainless steel, is used for the line on the filtrate side, wherein two glass windows or two sapphire glass windows are installed in the line at the location of measurement for the measurement by optical means.

In this connection, it should be pointed out again that only in the event that the virus filtration module has a defect, such as a crack, can a chain of air bubbles occur and be detected on the filtrate side. If no chain of air bubbles exits the virus filtration module, this means that there are no large or medium defects in it. In order to detect even the smallest defects, the (total) volume flow of air through the virus filtration module(s) is also measured. The (total) volume flow of air must not exceed 8 mL/min per virus filtration module.

Thus, the potentially present chain of air bubbles, which indicates a major to moderate damage or a major to moderate defect of the virus filtration module, can be optically recognized on the virus filtration module. Therefore the liquid serves only as a rinsing liquid and carrier in order to make for the chain of air bubbles in the carrier visible, as a result of which the testing of the virus filtration modules is possible. The integrity test is considered passed if no continuous chain of air bubbles is detected for a duration of at least 20 seconds over the entire test period and at the same time a (total) volume flow of air of at most 8 mL/min per virus filtration module is measured. The combination of both criteria (chain of air bubbles and measurement of volume flow of air) ensures that no defects can be overlooked in the tested virus filtration modules.

After the integrity test has been carried out, it may be expedient to dispose of the air and collect it, for example, in a waste collection container. The liquid, for example in the form of a buffer solution, can serve as a medium for subsequent virus filtration of a product.

According to one embodiment of the invention, the evaluation of the integrity test in step f) is also effected, in particular in an automated manner, with the specification for the integrity test, which requires that a continuous chain of air bubbles be present or not present for at least 20 seconds during the entire test period, and that a volume flow of air of maximum 8 mL/min per virus filtration module be measured or not. The test period begins at the moment when air is fed according to step d) and the selected test pressure of maximum 98+5 kPa is reached and lasts until the end of the test. Since step d) continues in the form of variants d1) or d2) during the carrying out of step e), the test period begins while step d) is being carried out and continues during step e). Based on the clearly defined and reproducible acceptance criteria, the test result of the integrity test can then advantageously be evaluated in an automated manner. This can be effected, for example, with the aid of software. Therefore, the result of the integrity test serves as a criterion for assessing the approval of the tested virus filtration module for virus filtration. Therefore, the following four cases are distinguished when testing the integrity of the invention from a virus filtration module:
a chain of air bubbles lasting at least 20 s is detected and the volume flow of air is above 8 mL/min→the virus filtration module is defective;
a chain of air bubbles lasting at least 20 seconds is detected and the volume flow of air is 8 mL/min or less→the virus filtration module is defective;
no chain of air bubbles is detected and the volume flow of air is above 8 mL/min→the virus filtration module is defective; or
no chain of air bubbles is detected and the volume flow of air is 8 mL/min or less→the virus filtration module is intact, there is no defect.

In this connection, it should be noted that the case in which a chain of air bubbles lasting at least 20 seconds is detected and the volume flow of air is 8 mL/min or less should not technically occur. This would mean that a medium to large defect would be present (chain of air bubbles lasting at least 20 s), but would not be recognized by the measurement of volume flow, which captures even the smallest defects. This case is only mentioned in order to list all (theoretically conceivable) test variants.

In the case of a non-integral virus filtration module, i.e., if a tested virus filtration module is not intact, i.e., does not pass the integrity test, the introduced air is not retained inside the hollow fibers due to damage to the hollow fiber, such as cracks. If there is major to moderate damage, the air bubbles pass via the filtrate side to a detection apparatus, in particular a scattered light sensor, which then detects a corresponding absorption signal for the air bubbles. If a chain of air bubbles occurs, the measured maximum volume flow of air is regularly above 8 mL/min per virus filtration module, usually significantly above 8 mL/min per virus filtration module, for example above 150 mL/min.

If there is a small or very small damage, it may be that no chain of air bubbles occurs, but the volume flow of air per virus filtration module is still above 8 mL/min, so that in this case as well the virus filtration module is recognized as not intact.

In the case of intact virus filtration modules, i.e., if a tested virus filtration module is intact, i.e., passes the integrity test, the fed-in air is retained due to the filter wetting and the pore size in the hollow fibers, which means that no corresponding absorption signal for a chain of air bubbles is detected on a detection apparatus, in particular a scattered light sensor, on the filtrate side of the filter module. The measured (maximum) volume flow of air is at most 8 mL/min per virus filtration module. Therefore, both criteria for passing the integrity test are fulfilled.

The integrity test according to the present disclosure therefore ensures that the hollow fibers of the virus filtration module are undamaged. While detection of the chain of air bubbles can only identify medium and large defects, the measurement of the volume flow of air through the virus filtration module(s) also makes it possible to capture even the smallest defects in the virus filtration module(s). Compliance with both criteria ensures the integrity of the virus filtration module(s). The check of the module is effected in a first integrity test (pre-visual leak test) prior to actual use (virus filtration) of the virus filtration module, in order to ensure that the module is suitable for use and is properly functioning. In addition, a second integrity test (post-visual leak test) is effected after the module has been used, i.e., after virus filtration, in order to ensure that it has not been damaged during virus filtration, for example due to overpressure or mechanical stress. It is understood that the first integrity test (pre-visual leak test) and the second integrity test (post-visual leak test) use the same method with the same procedure.

The detection of step e) and also the evaluation of step f) are therefore effected in particular in an automated manner, for example via a process control system (PCS), i.e., a computer system for controlling a method-related apparatus, as is known from the prior art. For example, the detection apparatus, such as a scattered light sensor, and a measuring apparatus for the volume flow of air are integrated into an internal process control system (PCS) and report within the running test program (steps a) to e) or f)) whether or not the defined acceptance criteria for passing the integrity test (no continuous chain of air bubbles for a duration of at least 20 seconds over the entire test period and measurement of the (total) volume flow of air through the virus filtration module(s) of at most 8 mL/min per virus filtration module over the entire test period) have been met. The use of a PCS system is particularly advantageous, since it ensures data integrity and the immutability of the recorded data and allows access to it at any time.

For example, a process control system used is part of a method-related filtration apparatus in which virus removal is carried out during a filtration controlled by the PCS. In the very same filtration apparatus, not only are the two integrity tests—an integrity test of the virus filtration module prior to virus filtration and an integrity test of the virus filtration module after virus filtration—carried out, but the virus removal from a product is also carried out between the integrity tests, without the need to install and remove the virus filtration module in a suitable apparatus for each of the methods. According to this embodiment, the functional testing of the virus filtration module(s) for filtration and the actual virus filtration with the virus filtration module(s) takes place in the same filtration apparatus.

According to a further embodiment of the invention, the integrity test of a virus filtration module is thus carried out in a filtration apparatus in which virus filtration is carried out between the two integrity tests. As a result, repeated installation and removal of the virus filtration module in a separate test system, along with installation and removal in a virus filtration system, are no longer necessary. The elimination of installation and removal steps has great advantages, since virus filtration modules are very sensitive to damage. For example, improper handling of a filter module, such as, for example, even a slight shaking, could result in the hollow fibers contained therein being destroyed. According to the invention, the virus filtration module(s) no longer need to be tested separately outside of a virus filtration system, as a result of which the risk of potential damage or contamination of the modules is significantly reduced. Therefore, the integrity test and virus filtration can be carried out in particular within the very same filtration apparatus.

The test procedure according to the invention for a virus filtration module can therefore be carried out by means of a partially or even fully automated method, wherein the method steps b) to d) and optionally also f) or the method steps a) to d) and, if applicable, also f) can each be carried out in a partially or fully automated manner. Step e) is always effected in a fully automated manner.

The test procedure of the invention represents a validatable method with which documented proof that the method continuously fulfills the defined specifications and quality characteristics is possible. Another advantage is that non-changeable parameters can be used in the test procedure, which can simplify the management of the method, for example by using a PCS system.

In particular, steps a) to e) or a) to f) are effected directly one after the other and in the specified order without any intermediate steps.

According to the present invention, one, two, three, four, five, six, seven or more virus filtration modules can be tested simultaneously. When carrying out the integrity test according to steps a) to e) or a) to f) with a plurality of hollow fiber filter modules simultaneously, parallel testing is carried out in particular. As a result, the time required per test for a filter module is significantly reduced. If one virus filtration module is tested, it takes approximately 10 to 15 minutes. If ten virus filtration modules are tested in parallel, it still takes 10 to 15 minutes, whereas testing each module individually would take 10 times as long. Only if testing a plurality virus filtration modules detects that one of them is not intact will individual testing be undertaken. This has the advantage of being a time-saving procedure, since a plurality of modules can be tested simultaneously to see whether they are fully functional.

It is expedient to carry out all steps in the same way for all virus filtration modules. For example, if a medium such as liquid or air is fed, it is fed from the same side in all virus filtration modules. If an optional intermediate step is carried out, it is carried out in parallel for all virus filtration modules; or this intermediate step is omitted for all.

The test period for the integrity test for detecting a chain of air bubbles, during which the volume flow of air continues to be measured, is at least 20 seconds. For example, a period of 20 seconds for the integrity test is generally considered sufficient by the manufacturers of virus filtration modules. However, testing can also be carried out for longer, for example 500 s or 400 s, in particular 300 s. This only represents the period during which the air is injected into the filter module and checked to see whether a continuous chain of air bubbles occurs for a duration of at least 20 seconds and at the same time the (total) volume flow of air is measured. In addition, there is the preparation and follow-up of the test, so that the integrity test for one tested filter module or per parallel tested filter module batch takes approximately 10 to 15 minutes. Due to possible bacterial growth, it is also expedient if the time interval between integrity testing and virus filtration does not exceed 24 hours, in particular less than 24 hours.

The invention also relates to a filtration apparatus for carrying out the method according to the invention, comprising
one, two, three, four, five, six, seven or more virus filtration modules in the form of hollow fiber filter modules, in each case comprising hollow fibers having an inner region and hollow fiber interstices, in each case having a feed side, a retentate side and a filtrate side;
devices for attaching and fastening one or more virus filtration modules;
in each case, one line on the feed side, one line on the retentate side and one line on the filtrate side of each virus filtration module, for feeding liquid and/or air at a constant pressure of at most 98±5 kPa to the virus filtration module(s);
opening and closing devices for the respective lines;
a connector for feeding liquid at a constant pressure of at most 98±5 kPa having the lines to the virus filtration module(s);
a connector for feeding air of a constant pressure at the same level as the liquid pressure having the lines to the virus filtration module(s);
a detection apparatus for automatically detecting a chain of air bubbles in the line on the filtrate side of the virus filtration module(s) and
a measuring apparatus for measuring the volume flow of air through the virus filtration module(s).

The explanations of the test procedure of the invention apply equally to the filtration apparatus.

The devices for attaching and fastening a virus filtration module in a filtration apparatus are not further restricted and are known to a person skilled in the art.

If a plurality of virus filtration modules are to be tested simultaneously, it is advantageous if they are aligned parallel to one another. For example, the hollow fibers of each filter module are arranged to run in particular in a vertical direction or in an intermediate position between vertical and horizontal position. A different orientation is also possible.

When testing a plurality of virus filtration modules simultaneously, it is expedient for method-related reasons if the liquid is fed to the filter module from the same side (feed side or retentate side) for all virus filtration modules tested simultaneously.

When testing a plurality of virus filtration modules simultaneously, it is also expedient if the air is fed to the filter module from the same side (feed side or retentate side) in each case via a line assigned to each virus filtration module, since, as a result, the virus filtration modules can be tested simultaneously, i.e., in parallel, and thus time can be saved.

When testing a plurality of virus filtration modules simultaneously, the respective lines on the filtrate side can be combined into one line, and the detection apparatus can then be arranged on the combined line. As a result, it is sufficient if one detection apparatus is used for a plurality of virus filtration modules. As a result, a complex measurement using a plurality of detection apparatuses in each of the lines on the filtrate side is avoided.

For detection purposes, a counting apparatus for the air bubbles of the chain of air bubbles, such as an air bubble counter, can also be provided. Additionally, an evaluation unit can be provided. The detection apparatus and evaluation unit are operated in particular in a fully automated manner and, for example, using software.

According to one embodiment, the filtration apparatus of the invention is part of a virus filtration system.

According to one embodiment, the filtration apparatus is designed so that one, two, three, four, five, six, seven or even more virus filtration modules can be subjected to the integrity test simultaneously, then a virus filtration can be carried out with them and subsequently the virus filtration module(s) can be subjected to the integrity test once more. In particular, virus filtration is carried out at the same or lower pressure selected in the integrity test.

The invention further relates to a method for cultivating prokaryotic or eukaryotic cells in liquid cell culture in a bioreactor to produce a product, in particular a protein, and carrying out virus filtration of the product obtained using one or more virus filtration modules, wherein the method for testing the integrity of the one or more virus filtration modules according to the present invention is carried out prior to and/or after virus filtration, in particular prior to and after virus filtration.

The invention also relates to a method for producing a recombinant protein, wherein the method comprises the following steps:
step a) cultivating prokaryotic or eukaryotic cells expressing a recombinant protein in cell culture in a bioreactor;
step b) harvesting the recombinant protein;
step c) purifying the recombinant protein;
step d) removing any viruses that may be present from the obtained recombinant protein in a virus filtration using one or more virus filtration modules, wherein the method for testing the integrity of the one or more virus filtration modules according to the present invention is carried out prior to and/or after virus filtration, in particular prior to and after virus filtration.

The product that is subjected to virus filtration according to the invention is not further restricted. According to one embodiment, it is a protein. The protein can, for example, be selected from the group consisting of monoclonal antibodies, polyclonal antibodies, monospecific antibodies, multispecific antibodies, single-chain antibodies or antibody fragments thereof, such as Fv, scFv, Fab, Fab′, scFab, F(ab′)2, Fab2, Fc and Fc′ fragments, immunoglobulin heavy and light chains and their constant, variable or hypervariable region along with Fv and Fd fragments, diabody, triabody, scFv-Fc, minibody or single-domain antibodies or a mixture thereof. In particular, the protein can be selected from a therapeutic protein used for preventing or treating a disease or disorder.

The advantages of the present invention are extremely complex:

The disadvantages of the previously known and used integrity tests are eliminated. Thus, the subjective visual air bubble check, carried out by at least one person at the end of the integrity test, is replaced by a detection method that is fully automated. The detection of a chain of air bubbles is advantageously effected by optical means, for example via a scattered light sensor that can be operated automatically.

The result in the form of two defined acceptance criteria for the integrity test, according to which the integrity test is passed if no continuous chain of air bubbles is detected for a duration of at least 20 seconds over the entire test period and, in particular, if a volume flow of air of at most 8 mL/min per virus filtration module is present, can be clearly captured and recorded more reproducibly. This makes possible the use of software that can carry out automatic detection and, if applicable, automatic evaluation of the integrity test.

The combination of the two criteria for passing the integrity test makes it possible to detect even the smallest defects in the virus filtration module(s).

Determining the volume flow of air even makes possible a quantitative evaluation of the number of defective hollow fibers. Capturing the air volume during the test phase and comparing it with an expected value of 8 mL/min per virus filtration module makes it possible to exclude diffusion effects.

In particular, method steps b) to d) or method steps a) to d) can be carried out in at least a partially automated manner. Step e) is always effected in a fully automated manner. The evaluation of step f) is also carried out in a particularly automated manner.

Due to the automation, the risk of human error is generally ruled out. The test results obtained are traceable and reproducible at any time due to data recording and thus meet the current requirements of data integrity.

As a result, no employees are required for the automated test procedure with defined parameters, or for capturing and evaluating the results. This comprises both the carrying out of the integrity test and can also extend to automated evaluation using suitable software.

According to the invention, the integrity test of a filter is effected using a validatable method, wherein non-changeable parameters can be used. Automation ensures the reliability and reproducibility of the method. In particular, by using a computer program in the form of a process control system (PCS), the management of the method can be simplified and carried out reliably.

For carrying out integrity tests on virus filtration modules, it is no longer necessary to provide a separate test apparatus into which the filter modules must be installed and removed for each test. Rather, it is now possible for the integrity test of the virus filtration modules to be effected in a filtration apparatus, with which the virus filtration can also be carried out. As a result, additional installation and removal steps are eliminated, along with potential sources of contamination and possible damage to the virus filtration modules. The virus filtration modules can advantageously be subjected to the integrity test(s) in the installed state, within a used filtration apparatus.

Manual handling of the virus filtration modules between tests, which carries a high risk of damage, is thus avoided and replaced by automated procedures.

One, two, three, four, five, six, seven or more virus filtration modules can be tested simultaneously and in parallel. This has the advantage that the time required per test for a filter module can be significantly reduced. A time-saving approach is of enormous advantage, in particular for a large-scale industrial process.

Only if a defect is detected during the simultaneous testing of a plurality of virus filtration modules in the integrity test (first integrity test or pre-visual leak test), then an individual test of the virus filtration modules is initially carried out in accordance with steps b) to e) or f). The defective virus filtration module(s) can then simply be replaced and the method can then be repeated according to steps b) to e) or f) with the replaced or all virus filtration modules. If a defect was already detected during the first integrity test, it may be useful, after the defect has been remedied by replacing the virus filtration module(s) and the first integrity test (pre-visual leak test) has been successfully carried out, to carry out the second integrity test after virus filtration (post-visual leak test) again in individual testing with the virus filtration modules in order to be able to better verify the result.

When testing a plurality of virus filtration modules simultaneously, the respective lines on the filtrate side can be expediently combined into one line, and the detection apparatus, such as a scattered light sensor, can be arranged on the combined line. As a result, only one detection apparatus is required for a plurality of filter modules, which significantly reduces the equipment required. This also means a significant simplification and acceleration of the detection and evaluation of the test.

According to one embodiment, the filtration apparatus of the invention is part of a virus filtration system, so that both the integrity tests and the virus filtration can be carried out in the very same filtration apparatus.

The present invention thus makes simplified and facilitated integrity testing of virus filtration modules possible. This is all the more true since the integrity test is regularly carried out twice with each virus filtration module, once before and once after the filter module is used. These advantages become even more apparent when a plurality of virus filtration modules are used simultaneously for testing and virus filtration.

In the following, the disclosure will be explained and illustrated in detail with reference to the accompanying drawings, without limiting the disclosure herein. Reference is made in detail herein to various embodiments of the invention, which are illustrated in the accompanying drawings. Wherever possible, the same or similar reference marks and symbols are used throughout the drawings to refer to the same or similar parts. The various elements shown in the drawing are representative only and not necessarily drawn to scale. The drawings are intended to illustrate only exemplary embodiments of the disclosure of the invention, and a person skilled in the prior art will readily recognize where the drawings have been simplified to illustrate key aspects of the disclosure.

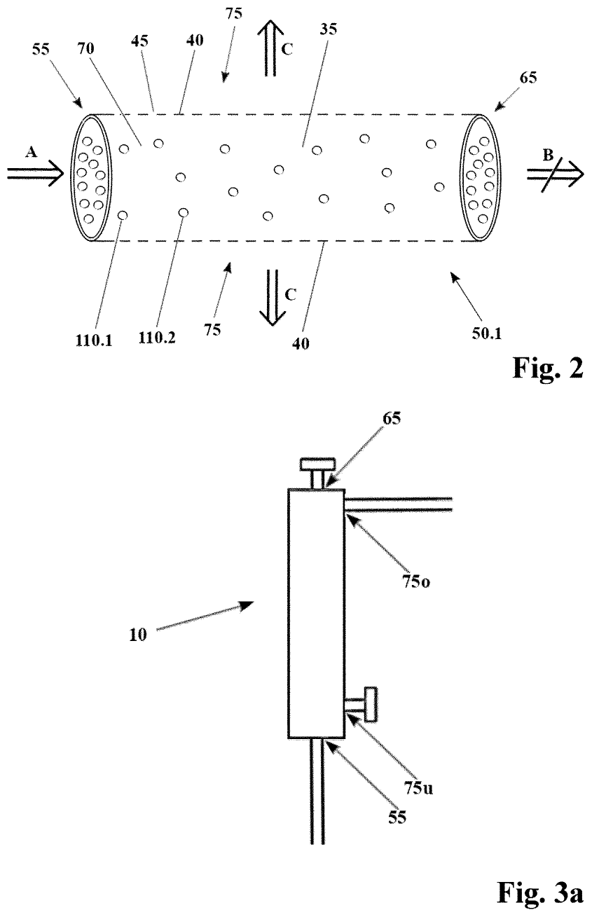

FIG. 1 to 3c have already been explained in the introduction to the description.

FIG. 4 is a schematic sectional view of a virus filtration module used according to the invention in the form of a hollow fiber filter module 10. The hollow fiber filter module 10 has a feed side 55, a retentate side 65 and a filtrate side 75 along with lines connected thereto, i.e., a line 55a that is connected to the feed side 55 of the hollow fiber filter module 10, a line 65a that is connected to the retentate side 65 of the hollow fiber filter module 10 and a line 75a that is connected to the filtrate side 75 of the hollow fiber filter module 10. The hollow fiber filter module 10 has a bundle 50 of hollow fibers, which comprises, among others, the hollow fibers 50.1 and 50.2. As can be seen from FIG. 4, the hollow fibers 50.1, 50.2, . . . are arranged in the filter module 10 and have such an orientation that the hollow fibers 50.1, 50.2, . . . run in the vertical direction from the feed side 55 of the filter module 10 to the retentate side 65 of the filter module 10, wherein the hollow fibers 50.1, 50.2, . . . are also substantially parallel to one another.

The vertical position shown in FIG. 4 is shown here solely to simplify the explanations. Other orientations are possible, such as an intermediate position between the vertical and horizontal positions. By way of example, FIG. 5 shows an intermediate position of the virus filtration module 10, which is located between a vertical axis V and a horizontal axis H. The virus filtration module 10 is in an (arbitrary) inclined position that deviates more or less strongly from the vertical direction, so that the alignment of the hollow fibers does not run parallel to the axis V. The virus filtration module 10 is not in an exactly upright or vertical position and is also not in an exactly lying, i.e., horizontal, position with the hollow fibers running parallel to the horizontal axis H.

There are interstices (not shown) between the hollow fibers 50.1, 50.2, . . . In step a) of the test procedure according to the invention, the virus filtration module 10 is therefore installed in a filtration apparatus in an upright position with vertically running hollow fibers 50.1, 50.2 . . . , as shown in FIG. 4. Other alignments of the hollow fibers 50.1, 50.2 . . . are possible.

A filter module 10 is used, which is approved for a maximum pressure of up to 98±5 kPa. If higher pressure is applied, the filter module 10 could be destroyed. A filter module that can be used for higher pressures is not used according to the invention.

Prior to carrying out step a), it may be expedient to carry out sterilization of the lines 55a, 65a and 75a, in particular by hot steam sterilization. This is usually carried out without a virus filtration module, wherein instead of the virus filtration module, for example, a fitting piece is inserted into the filtration system for closing the open section. A fitting piece is, for example, an intermediate line piece. Sterilization (SIP, sterilization in place) can be effected, for example, at 100° C. or above, particularly in the range of 100-130° C. This is useful if the integrity test is followed by virus filtration and a sterile product is to be obtained. Following sterilization of the lines, the lines can then be placed under overpressure, for example of approximately 100 kPa, in order to prevent the entry of contamination after sterilization. If overpressure is used, a pressure reduction is effected in all lines before the integrity test is carried out, for example to a pressure P≤10 kPa, wherein a pressure equalization is effected throughout the system. This serves to protect the virus filtration module from damage.

The lines 55a, 65a and 75a are in each case made, for example, of plastics material or metal, in particular stainless steel. It may be advantageous if all lines of the virus filtration module 10 are selected from the same material; however, this is not necessary in every case.

In step b) of the method, the virus filtration module 10 and the connected lines 55a, 65a and 75a are filled with liquid 70 at a constant pressure of at most 98±5 kPa in 3 sub-steps. If applicable, any additional inlets and outlets of the virus filtration module 10 that are not used for the integrity test are closed. An unused outlet of the virus filtration module 10 is shown in FIG. 4 by the line 75b on the filtrate side 75 of the filter module 10, which is closed. This can be effected, for example, by means of a valve, a plug, a corresponding clamp or in another way.

Initially, in step b1), all lines 55a and 65a connected to the virus filtration module are filled with liquid 70, but without the virus filtration module 10. In step b2), the inner region of the hollow fibers 50.1, 50.2 . . . of the virus filtration module 10 is filled with liquid 70. This is done by opening valve 55v of line 55a on the feed side 55 and valve 65v of line 65a on the retentate side 65 and closing valve 75v of line 75a on the filtrate side 75. In step b3), the interstices between the hollow fibers 50.1, 50.2 . . . of the virus filtration module 10 are then filled with liquid 70. Here, either the line 55a on the feed side 55 and the line 75a on the filtrate side 75 are opened, and the line 65a on the retentate side 65 is closed. Alternatively, the line 65a on the retentate side 65 and the line 75a on the filtrate side 75 will be opened and the line 55a on the feed side 55 will be closed.

The liquid 70 used is not particularly restricted; this depends on the subsequent location and purpose of the filter module 10. For example, a liquid including water, in particular water as the main component, is used. This can also be a water-based buffer solution. The purity of the water depends on the intended use, in particular from which liquid product-containing composition the viruses are subsequently to be removed.

The filling with liquid 70 in steps b2) and b3) can be effected in each case either from the feed side 55 (in FIG. 4 from below) via the line 55a or from the retentate side 65 (in FIG. 4 from above) via the line 65a. Filling with liquid 70 from the feed side 55 is particularly advantageous, since this is generally simpler in terms of method technology and is therefore particularly easy to carry out. A further advantage of feeding the liquid 70 from below is that any air present in the filter module 10 is thereby forced upwards out of the filter, so that no more air can remain in the filter. At the same time, a complete venting of the filter module 10 is achieved.

The expression of feed “from below” or “from above” or similar formulations does not mean that a filter module 10 must always assume an exactly vertical position. Rather, the filter module 10 can also assume any intermediate position deviating from the vertical alignment, which lies between a vertical position (hollow fibers parallel to a vertical axis) and a horizontal position (hollow fibers parallel to a horizontal axis) (see FIG. 5). Even if the filter module 10 is inclined at an angle away from the vertical direction, the feed can be from below or above.

The filling level of the virus filtration module 10 in steps b2) and b3) can be measured by measuring the pressure prior to and after the filter module, in FIG. 4 for example in the lines 55a and 65a for step b2) and in the lines 55a and 75a for step b3) and determining the differential pressure in each case. At a maximum pressure difference of 98±5 kPa, it can be assumed that the virus filtration module is filled according to step b2) or b3). Alternatively or additionally, the amount of liquid fed can be determined and set in relation to the dead volume of the lines 55a, 65a and 75a and the virus filtration module 10, wherein the amount of liquid ≥dead volume. The dead volume in FIG. 4 would be the volume of the lines 55a, 65a, 75a and the filter module 10 without liquid. This can be determined in each individual case or inquired with the manufacturer.

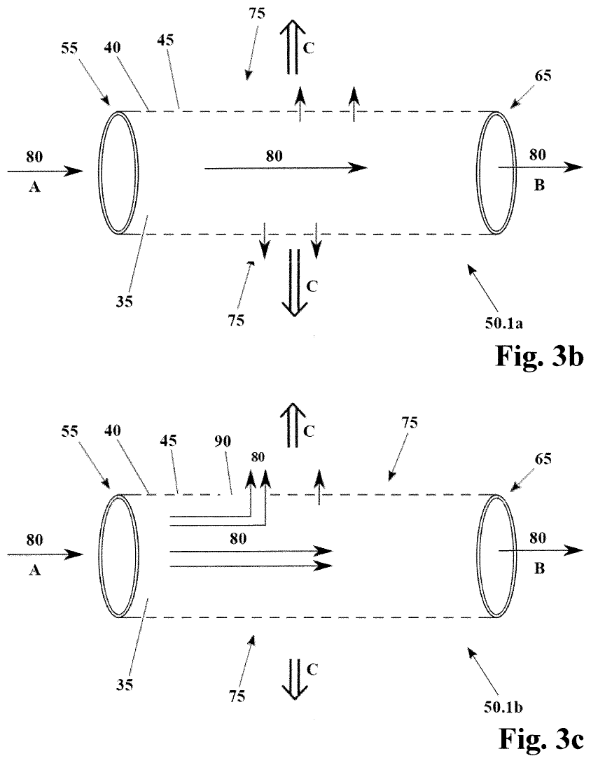

Accordingly, in FIG. 4, the hollow fiber filter module 10 is completely filled with liquid 70, so that—to the extent physically possible—no empty space remains in the filter module 10. In other words, all inner regions of the hollow fibers (e.g., inner region 35 of the hollow fiber 50.1a in FIG. 3b) along with all interstices between the hollow fibers 50.1, 50.2, . . . (e.g., intermediate regions 38.1, 38.2 . . . in FIG. 3 b) are filled with liquid 70. Any air present is displaced by the liquid 70, so that the filter module 10 is also vented. The filter module 10 is thus completely filled with liquid 70. The virus filtration module and all lines are almost completely filled by adding the liquid 70 under pressure; the pressure is selected to be a maximum of 98±5 kPa. Lower pressure values are also possible. The higher the pressure is set, the more easily the air bubbles in a chain of air bubbles can be detected.

The three arrows A, B, C in FIG. 4 indicate the respective flow direction when filling the liquid 70 through the hollow fiber filter module 10 for the method alternative according to the invention, according to which the liquid 70 is fed in steps b2) and b3) from the feed side 55 to the filter module 10, by way of example. Other method variants are also possible.

Filling with liquid 70 is mandatory in the three sub-steps described, since otherwise the filter module 10 cannot be completely filled, as already explained.

Once the hollow fiber filter module 10 and the lines 55a, 65a and 75a are completely filled with liquid, the feed of liquid 70 is stopped and all lines 55a, 65a, 75a are closed, wherein the pressure (step c) is maintained. The closure of the lines 55a, 65a, 75a is effected by means of suitable devices known from the prior art to a person skilled in the art. These are, for example, valves 55v, 65v and 75v, which are provided in the respective lines. Other opening and closing devices are also possible. As a result, a closed system completely filled with the liquid 70 is obtained, which remains under the applied pressure.

Step c) may be followed by an optional intermediate step in which the inner region of the hollow fibers 50.1, 50.2 . . . of the virus filtration module 10 and the line connected thereto, line 55a in FIG. 4, are again completely emptied of liquid 70. This can be a particularly gentle procedure for the virus filtration module 10 and protect the module from damage. This has no adverse effects on the subsequent integrity test.

After step c) or after the optional intermediate step, but prior to carrying out step d), a leak test of the complete system, including the virus filtration module, can be carried out in order to rule out any leaks in the system. This serves to ensure that no air escapes from the system, as a result of which the subsequent integrity test and its evaluation could be impaired. However, since the volume flow of air through the filter module 10 is measured during the integrity test, in particular during steps d1) or d2), a leak test can generally be omitted. This will only be described here for the sake of completeness.

In the leak test, air is forced into the system. In FIG. 4, this can be effected either from the retentate side 65 or from the feed side 55 of the virus filtration module 10. For example, the air pressure is increased step-by-step from 0 kPa to 70 kPa. 70 kPa then represents the pre-stabilization pressure. After reaching the pre-stabilization pressure, a stabilization period of at least 180 s is maintained, during which the air can be dispersed in the system prior to the evaluation of the leak test. If the pressure drop after the stabilization period is ≤10 kPa, the leak test is passed. If the pressure drop is >10 kPa, there is a leak in the filtration system, which must be eliminated.

Air is subsequently fed to the virus filtration module with step d1) or d2). Here, a selection is made between two method alternatives: either step d1) or step d2) is followed, depending on the line through which the air is fed to the virus filtration module 10:

In the first method variant, in step d1), the line 55a on the feed side 55 and the line 75a on the filtrate side 75 of the filter module 10 are opened and the line 65a on the retentate side 65 is closed or remains closed. Other lines not used for the test, here: line 75b on the filtrate side 75 of the filter module 10, remain closed. The selected pressure of at most 98±5 kPa is maintained throughout the integrity test and thus also during the carrying out of step d1). In step d1), air with a constant pressure at the same level as selected for the liquid pressure is fed from the line 55a on the feed side 55 into the virus filtration module 10. For this purpose, for example, a connector (not shown) is provided on the line 55a in order to inject air into the line 55a. The injected air has a pressure that is exactly the same as the pressure selected for the liquid 70. For example, a pressure is set for the liquid and the air, which in each case is 98±5 kPa. This has the advantage that the chain of air bubbles, if formed, is particularly easy to detect.

Alternatively, in the second method variant, in step d2), the line 65a on the retentate side 65 and the line 75a on the filtrate side 75 of the filter module 10 are opened and the line 55a on the feed side 55 is closed, if this has not already been effected. Other lines not used for the test, here: line 75b on the filtrate side 75 of the filter module 10, remain closed. The selected pressure of a maximum of 98±5 kPa is maintained throughout the integrity test and thus also during the carrying out of step d2). In step d2), air with a constant pressure at the same level as selected for the liquid pressure is fed from the line 65a on the retentate side 65 into the virus filtration module 10. For this purpose, a connector (not shown) is provided on the line 65a in order to inject air into the line 65a. The injected air has a pressure that is exactly the same as the pressure selected for the liquid 70. For example, a pressure is set for the liquid and the air, which in each case is 98±5 kPa. This has the advantage that the chain of air bubbles, if formed, is particularly easy to detect.

In the method according to the invention, unlike most systems from the prior art, there is no pressure difference; rather, the same pressure of maximum 98±5 kPa is present everywhere. Surprisingly, transmembrane pressure is not required. Setting a pressure difference is generally disadvantageous, since an exact pressure difference cannot be set exactly; there will always be a range of fluctuation (comparable to the speed setting in cruise control). It is assumed that fluid mechanics plays a role in this; adhesion processes are only of minor importance at best. The integrity test can also be carried out at low pressures of 10±0.5 kPa or 20±1 kPa, since, as tests show, a continuous chain of air bubbles can be detected in this case as well.

In method steps d1) and d2), air is thus injected into the filter module 10. This air is used to detect whether the hollow fibers in the filter module 10 are intact, i.e., they do not exhibit any damage, such as cracks, and therefore fulfill the desired filter function. When the filter module is functional, the air is retained inside the hollow fibers 50.1, 50.2 . . . , since the air cannot penetrate the hollow fiber wall. The incoming air is therefore forced via the selected filter inlet of the virus filtration module 10 through the hollow fibers 50.1, 50.2 . . . to the filter outlet. In this case, the filter inlet is the side of the filter module 10 where the air is introduced, the filter outlet is the side of the filter module 10 where the air is supposed to exit again when the hollow fibers are intact.

If the air is injected into the virus filtration module 10 from below via the line 55a, the air exits via the line 65a if the virus filtration module 10 is intact. Since it is a closed system, the air is dispersed between the line 55a, the filter module 10 and the line 65a. If the air is injected into the virus filtration module 10 from above via the line 65a, the air exits via the line 55a if the virus filtration module 10 is intact. Since this is a closed system, the air is also dispersed between the line 55a, the filter module 10 and the line 65a in this case as well. In the case of a non-functional filter module 10, the air passes through damages in the hollow fiber wall and thus reaches the filtrate side 75 and thus into the line 75a of the filter module 10, where a chain of air bubbles then occurs and can be detected in the line 75a. At the same time, the volume flow of air or the maximum volume flow (Volₘₐₓ) of the air through the filter module 10 is measured with a measuring apparatus (not shown). The volume flow of air must not exceed 8 mL/min for a functional filter module 10. Therefore, the following cases are distinguished when testing filter module 10:
Volₘₐₓ is above 8 mL/min+chain of air bubbles ≥20 s or
Volₘₐₓ is at most 8 mL/min+chain of air bubbles ≥20 s or
Volₘₐₓ is above 8 mL/min+chain of air bubbles ≤20 s.

In these three cases, the filter module 10 is not functional and contains a defect.
Volₘₐₓ is at most 8 mL/min+chain of air bubbles ≤20 s.

In this case, the filter module 10 is functional and does not contain any defects.

In this connection, it should be noted that it is assumed that the case “Volₘₐₓ is at most 8 mL/min and the chain of air bubbles ≥20 s” cannot occur. This would mean that a medium to large defect would be present (chain of air bubbles ≥20 s), but would not be recognized by the measurement of volume flow, which captures the smallest defects. This case is only mentioned in order to list all (theoretically conceivable) test variants. This case has no practical significance.

The second method alternative, according to which the air is injected into the virus filtration module 10 from above via the line 65a, has proven to be particularly advantageous, in particular if air is introduced into the filter module 10 from the highest point thereof.

The air used is not particularly restricted. It is understood that it should not contain any components that could in any way impair the virus filtration module 10 or the integrity test. In particular, process air is used.

According to one embodiment, the air used in method steps d1) or d2) is in each case initially passed through an air filter (not shown in FIG. 4, but in FIGS. 5a and 5b) before it can enter the hollow fiber filter module 10 in order to retain potential contaminants from the air so that they do not contaminate the filter module 10. It has been found that the test procedure is not impaired by such an air filter.

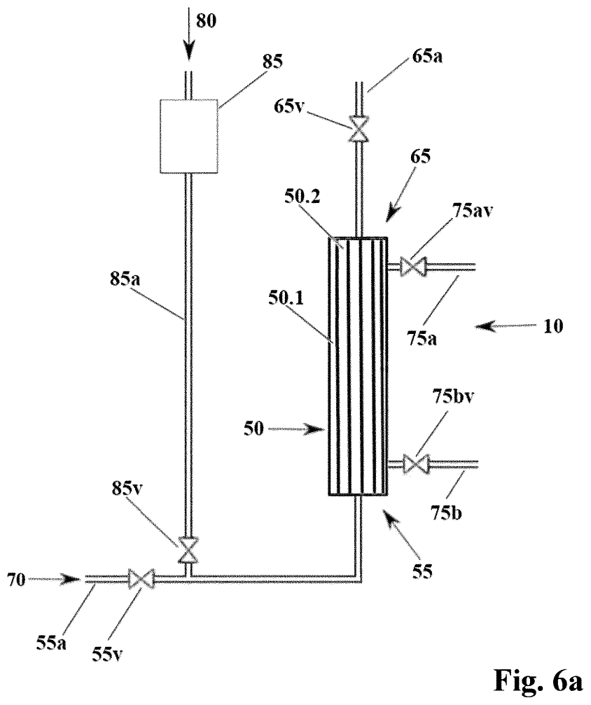

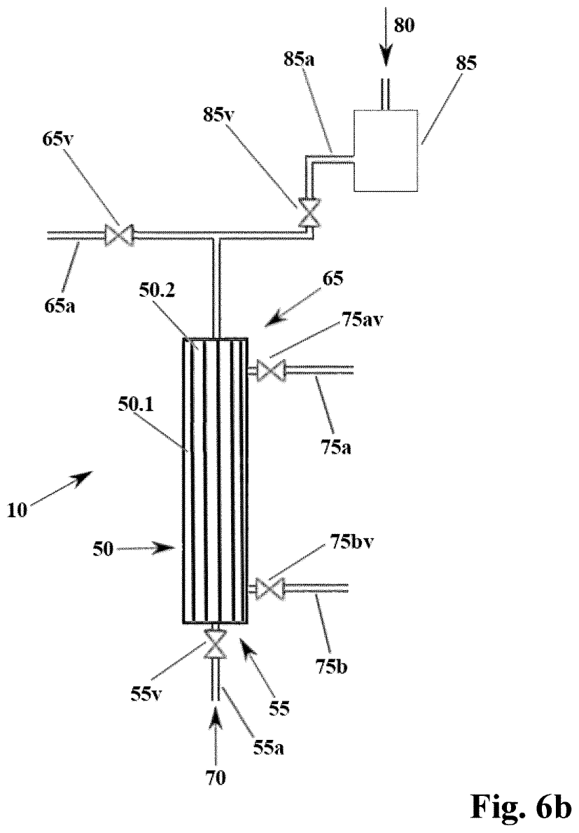

FIGS. 6a and 6b are a schematically simplified view of a virus filtration module in the form of a hollow fiber filter module 10 with connected lines and the relative position of an air filter 85 to the hollow fiber filter module 10. FIG. 6a illustrates an exemplary embodiment in which the virus filtration module 10 as a whole and its lines 55a, 65a and 75a are already completely filled with liquid (steps b1) b2) and b3)). In the example shown, the liquid 70 was fed to the filter module 10 from below, i.e., through the valve 55v and via the line 55a. Alternatively, the liquid 70 could also be fed from above, i.e., through the valve 65v and via the line 65a to the filter module 10. The valves 55v, 65v, and 75av in FIG. 6a were closed after filling; the valves 75bv and 85v were already closed prior to filling. It is therefore a self-contained system.

For the sake of simplicity, the virus filtration module is always shown in a vertical position in the figures. However, this is not necessary in practice. The virus filtration module can be installed in any alignment in a filtration apparatus.

In the example shown, the air should now be fed from below via the line 55a. For this purpose, the valve 85v is opened so that liquid 70, which is located in the line 55a, can enter the line 85a. In the example shown, the air filter 85 is arranged above the filter module 10. As a result, the fact that the air filter 85 is located entirely above the filter module 10 simply prevents the liquid 70 from rising out of the liquid-conducting line 55a through line 85a to the air filter 85 and, if applicable, impairing its function.

Alternatively, it would already be sufficient if the air-conducting line 85a leading from the air filter has a portion that comprises the highest point of the filtration apparatus. Then the air filter 85 could be arranged next to or below the virus filtration module 10. As a result, it would be ensured that liquid 70 cannot rise up to the air filter 85 and penetrate into it.

In FIG. 6b, in the example shown, the air is now fed from above via the line 65a. For this purpose, the valve 85v is opened so that liquid 70, which is located in the line 65a, can enter the line 85a. In order to prevent this, in FIG. 6b the air filter 85 is arranged entirely above the filter module 10, so that the liquid 70 from the liquid-conducting line 65a cannot rise via the line 85a to the air filter 85 and, if applicable, impair its function. According to a further embodiment, the air-conducting line 85a leading from the air filter could also have a portion that comprises the highest point of the filtration apparatus. In this case, the air filter 85 could also be arranged below the virus filtration module 10. As a result, it is presented that liquid 70 rises to the air filter 85 and penetrates it.

After or during step d) and before step e) is carried out, according to one embodiment, a certain period of time can be waited for, during which the air is further fed and the air can be further dispersed in the system. This is the so-called stabilization period, which can be up to 600 s. However, this can also simply be omitted.

As shown schematically in FIG. 4, one of the two method alternatives d1) or d2), if applicable after a stabilization period of up to 600 s, is followed by step e), i.e., an automatic detection of a chain of air bubbles that may occur on the filtrate side 75 of the filter module 10 in the line 75a. Step e) takes place while step d1) or step d2) is still being carried out. At the same time, the volume flow of air through the virus filtration module 10 is measured with a measuring apparatus (not shown).

The detection of a possible defect in the virus filtration module 10 based on an occurring chain of air bubbles is advantageously effected by optical means. For example, a scattered light sensor 120 is used, as schematically illustrated in FIG. 4 in the form of two rectangles. The sensor 120 measures the light scattering (side scattering) and, as a rule, is constructed from a light source and one or more detectors for detecting the light after it has passed through the line 75a and the liquid contained in the line, which, if applicable, contains a chain of air bubbles. For example, light in the near infrared range having a wavelength in the range of 730 to 970 nm is emitted by a light source of the sensor 120, passes through the line 75a filled with liquid and is absorbed by the air bubbles of a chain of air bubbles present in the liquid. The light exiting the line 75a can be captured again, for example, by a detector such as a photocell, photodiodes or the like, and a measured value can be ascertained therefrom. Therefore, the scattered light sensor 120 measures the changed absorption of light if a chain of air bubbles occurs.

At the position where the detection of the chain of air bubbles takes place, in particular at the location of the detection or measurement, the line 75a on the filtrate side 75 is designed to be at least partially transparent. In FIG. 4, the location of detection or measurement is the part of the line 75a that is located between the individual components of the scattered light sensor 120 (symbolized by two rectangles). Since the detection of the chain of air bubbles in the embodiment shown is effected by optical means, at least those portions of the line 75a on the filtrate side 75 which are located in the beam path of the light used are selected to be transparent.

According to one embodiment, a transparent tube or two transparent portions in the form of two windows are used for the line 75a, which in each case is selected in its dimensions so that it does not obstruct the beam path of the entering and exiting light in the sensor 120, so that the light can pass through unhindered.

Transparent materials that may be considered have already been described. The thickness of the transparent material depends on its type and composition. This can, for example, be selected to be a few mm or even a few cm thick in order to withstand the selected pressure.

If two transparent windows are used, both are made of the same material and both have the same strength (=thickness).

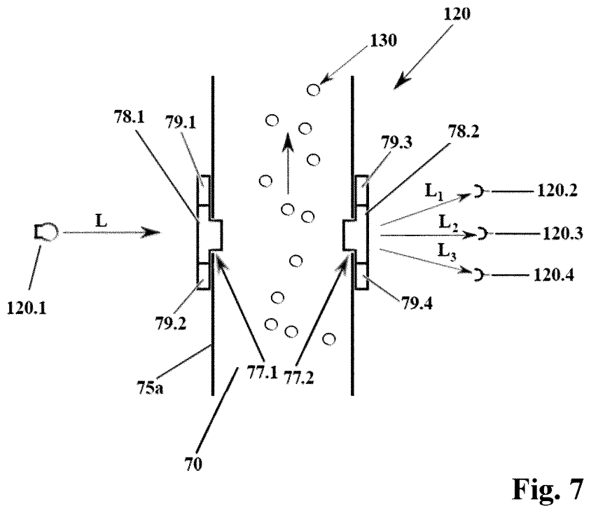

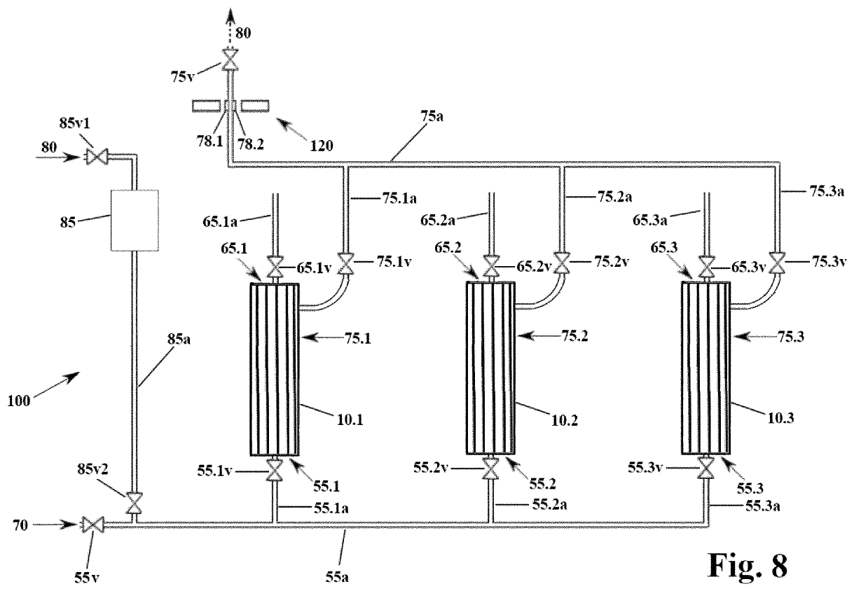

An exemplary setup for scattered light measurement with a scattered light sensor 120 is illustrated in FIG. 7 in a simplified form in a partial sectional view. A section through the line 75a filled with liquid 70 on the filtrate side 75 of the virus filtration module 10 is shown. The liquid 70 is located in the line 75a under the selected pressure. A light source 120.1, for example a tungsten lamp, emits light L of the desired wavelength, which is, for example, in the range from 730 to 970 nm. In the line 75a, at the location of the measurement, i.e., in the path of the light L to be measured, a transparent portion in the form of windows 78.1 and 78.2 is provided on the two opposite sides of the line 75a, so that the light radiated by the light source 120.1 passes through the first window 78.1 into the line 75a filled with liquid and then exits the line 75a again through the second window 78.2. The material of the windows 78.1, 78.2 is made, for example, of borosilicate glass, such as Pyrex®, or sapphire. The windows 78.1 and 78.2 close the openings 77.1 and 77.2 of the line 75a and cover them at the same time. In addition, seals 79.1, 79.2, 79.3 and 79.4 are provided in order to seal the line 75a.

If a chain of air bubbles 130 in the liquid 70 of the line 75a now travels through the beam path of the light L, the light L is absorbed and scattered in a corresponding manner (light beams L1, L2 and L3), which is captured with the aid of the detectors 120.2, 120.3 and 120.4 shown by way of example. These are, for example, photodiodes. The exact optics in the beam path of the light L prior to and after passing through the line 75a with various lenses and the like are not shown in FIG. 7 and are known to a person skilled in the art. For example, commercially available scattered light sensors, in particular well-known turbidity sensors, can be used.

Therefore, the scattered light sensor 120 is attached to the filter outlet on the filtrate side 75 of the virus filtration module 10 and detects a chain of air bubbles 130 occurring in the line 75a.

In addition to checking whether a continuous chain of air bubbles occurs, the volume flow of air through the virus filtration module 10 is measured.

The result of the integrity test then serves as a criterion for assessing a subsequent release of the virus filtration module 10 for virus filtration. If the virus filtration module 10 is intact, the volume flow of air of the virus filtration module 10 does not exceed the limit of 8 mL/min and the scattered light sensor 120 does not detect a corresponding absorption signal for a chain of air bubbles on the filtrate side 75 of the filter module 10. If the virus filtration module 10 is not intact, the volume flow of air of the filter module 10 exceeds the limit of 8 mL/min and, if applicable, the scattered light sensor 120 on the filtrate side 75 of the filter module 10 also detects an absorption signal for a chain of air bubbles.

The evaluation of the detection, in particular the scattered light measurement and measurement of the volume flow of air, is effected in step f) in particular also in an automated manner based on the specifications for the integrity test, that a chain of air bubbles is present for at least 20 s or not, and that the volume flow of air exceeds 8 mL/min per virus filtration module or not. According to one embodiment, the scattered light sensor 120 along with the measuring apparatus (not shown) for the volume flow of air are integrated into an internal process control system (PCS) (not shown) and report within an ongoing test program as to whether or not the defined acceptance criteria for passing the integrity test have been met.

For example, the process control system can be part of a method-related filtration apparatus in which integrity tests and virus removal can be carried out with the virus filtration module 10.

FIGS. 8 and 9 in each case are schematic sectional views of a section of a filtration apparatus 100 according to further embodiments of the invention, in which in each case both the integrity test and virus filtration can be carried out. In addition, a plurality of virus filtration modules can be tested simultaneously in the filtration apparatus 100 using the method according to the invention.

In detail, FIG. 8 is a section of a filtration apparatus 100 in which both the integrity tests having the virus filtration modules and virus filtration having them can be carried out. Three virus filtration modules are shown, in each case in the form of hollow fiber filter modules 10.1, 10.2 and 10.3 in installed state, in which the hollow fibers are oriented so that they run in a vertical direction (step a)). Of course, more than the virus filtration modules shown by way of example can be provided. This depends on the type and size of the filtration apparatus and the virus filtration to be carried out.

The vertical orientation was selected only for simplification and better explanation. Of course, another position can also be selected, such as any intermediate position between vertical and horizontal orientation (see FIG. 5).

The virus filtration modules 10.1, 10.2 and 10.3 in each case have a feed side 55.1, 55.2 and 55.3, a retentate side 65.1, 65.2 and 65.3 and a filtrate side 75.1, 75.2 and 75.3. Each filter module 10.1, 10.2 and 10.3 is connected to the lines 55.1a, 55.2a and 55.3a on the feed side, the lines 65.1a, 65.2a and 65.3a on the retentate side and the lines 75.1a, 75.2a and 75.3a on the filtrate side. A further line on the filtrate side of each virus filtration module 10.1, 10.2, 10.3 has been omitted for the sake of clarity.

The material of the lines is not particularly restricted; in FIG. 8 the lines are made of stainless steel.

For carrying out the integrity test, a liquid 70, in the example shown ultrapure water, in particular WFI water (WFI, water for injection), is used and the virus filtration modules 10.1, 10.2 and 10.3, which are located in parallel to one another, and all lines connected thereto with ultrapure water have already been filled with liquid 70 (steps b1), b2) and b3)). This can be carried out analogously to FIG. 4, but for all three virus filtration modules 10.1, 10.2 and 10.3 and all lines connected thereto simultaneously. According to the invention, the water 70 can be supplied either from below through the lines 55.1a, 55.2a and 55.3a on the feed side or from above through the lines 65.1a, 65.2a and 65.3a on the retentate side. The terms “from above” or “from below” should be understood as broadly as possible and can, for example, also refer to another intermediate position of the virus filtration modules 10.1, 10.2, 10.3, as already explained for a virus filtration module in connection with FIG. 5. In the embodiment shown in FIG. 8, the water 70 is fed in each case from below through the valve 55v via the lines 55.1a, 55.2a and 55.3a.

For method-related reasons, it is expedient if the water 70 is fed from the same side for all virus filtration modules 10.1, 10.2 and 10.3 tested simultaneously, in FIG. 8 in each case from below for all virus filtration modules. Alternatively, the water 70 could also be fed from above to all virus filtration modules.

In each of the lines 55.1a, 55.2a and 55.3a for feeding water 70, inlet filters (not shown) can be provided in order to remove possible impurities from the water before it enters the filter module. The water 70 is fed at a pressure of at most 98±5 kPa in 3 sub-steps-as already explained-so that all inner regions of the hollow fiber, all interstices between the hollow fibers and all connected lines on the virus filtration module 10.1, 10.2 and 10.3 are filled with water 70. The pressure range is selected in a range that does not impair the virus filtration modules 10.1, 10.2 and 10.3.

As soon as the virus filtration module 10.1, 10.2 and 10.3 and the respective lines 55.1a, 55.2a and 55.3a; 65.1a, 65.2a and 65.3a along with 75.1a, 75.2a and 75.3a are completely filled with water 70, the feed of water 70 is stopped (step c)) and all lines are closed. For this purpose, the valves on the feed side 55.1v, 55.2v, 55.3v, the valves on the retentate side 65.1 v, 65.2v, 65.3v and the valves on the filtrate side 75.1v, 75.2v, 75.3v are in each case closed. This results in a closed system completely filled with water 70, which is still under the applied pressure of at most 98±5 kPa.

In a subsequent optional intermediate step, the water 70 can be completely emptied from the inner region of each virus filtration module 10.1, 10.2 and 10.3 and the associated lines 55.1a, 55.2a and 55.3a. This serves as additional protection of the modules 10.1, 10.2 and 10.3 from damage. This intermediate step can also be omitted.

This can then be followed by a leak test (pressure hold test) of the lines of the filtration apparatus 100 and the installed virus filtration modules 10.1, 10.2 and 10.3. For this purpose, the air is injected into the virus filtration modules either from below or from above and the corresponding lines filled with liquid on each virus filtration module are opened. If air is fed from above, the lines 65.1a and 75.1a on virus filtration module 10.1 and the analogous lines on the other two virus filtration modules 10.2 and 10.3 are opened. Alternatively, if air is fed from below, lines 55.1a and 75.1a on virus filtration module 10.1 and the analogous lines on the other two virus filtration modules 10.2 and 10.3 are opened. The inlets and outlets not required of the system for water 70 (valve 55v) and air 80 (valve 75v) are closed. Air is then injected into the virus filtration modules either from above or from below. In FIG. 8, this would be effected from below from the feed side via the lines 55.1a, 55.2a and 55.3a into the virus filtration modules 10.1, 10.2 and 10.3 at the same pressure as the water pressure, wherein the air pressure is increased step-by-step from 0 kPa to a pre-stabilization pressure of, for example, 70 kPa. After an optional stabilization period of, for example, at least 180 s, it is detected whether the pressure drop is ≤10 kPa. If this is >10 kPa, there is a leak in the filtration system, which must be eliminated. However, since the volume flow of air is measured during the integrity test, in particular while steps d1) or d2) are still ongoing and step e) is being carried out, the leak test is superfluous and can be omitted. This is explained here for illustrative purposes only.

In the following step d), air is injected into the virus filtration modules 10.1, 10.2, 10.3; this occurs either from below (step d1)), in each case through the line on the feed side, or from above (step d2)), in each case through the line on the retentate side of each virus filtration module 10.1, 10.2, 10.3. In the embodiment shown in FIG. 8, the air 80 is injected from below according to step d1) of the method according to the invention. If a leak test is to be carried out, it is expedient if air is introduced from below, as is effected in step d).

In detail, according to step d1), the lines on the feed side for each virus filtration module and the lines on the filtrate side are opened and the other lines remain closed, wherein the pressure (as specified in step b)) is maintained, in order to feed air at a constant pressure at the same level as the water pressure from the line on the feed side (from below) into the virus filtration module. In FIG. 8, therefore, the lines 55.1a, 55.2a and 55.3a on the feed side 55.1, 55.2 and 55.2 (in each case at the lower end of the virus filtration module 10.1, 10.2, 10.3) and the lines 75.1a, 75.2a and 75.3a on the filtrate side 75.1, 75.2 and 75.3 of the filter module 10.1, 10.2 and 10.3 (at the side wall of the filter module 10.1, 10.2 and 10.3) are in each case opened and the line 65.1a, 65.2a and 65.3a on the retentate side 65.1a, 65.2a and 65.3a (at the upper end of the filter module 10.1, 10.2 and 10.3) remain closed. Opening the lines does not cause a pressure drop. Rather, the pressure remains constant throughout the entire test procedure at the pressure value used for the test procedure, which was selected from the range of at most 98±5 kPa.

The opening and closing of the lines can be effected by means of devices known to a person skilled in the art. Valves are shown in FIG. 8; other options are conceivable.

After opening the lines on the feed side and the lines on the filtrate side, air 80 is fed via the valves 85v1 and 85v2 into the lines 55.1a, 55.2a and 55.3a, which are located in each case on the feed side 55.1, 55.2 and 55.2 of the virus filtration modules 10.1, 10.2, 10.3, at a constant pressure of at most 98±5 kPa to the respective virus filtration modules 10.1, 10.2, 10.3. The air pressure is set to the same level as the previously set water pressure.

In FIG. 8, according to one embodiment, the air 80 is initially passed through an air filter 85 in order to remove impurities from the air so that they cannot enter one of the filter modules 10.1, 10.2 and 10.3. The air filter 85 is arranged above the virus filtration modules 10.1, 10.2 and 10.3. Alternatively, the air-conducting line 85a leading from the air filter 85 could have a portion that comprises the highest point of the filtration apparatus, so that no water can rise to the air filter 85, even if the air filter 85 were not arranged above the virus filtration modules 10.1, 10.2, 10.3. Other embodiments are also possible. As a result of the fact that the air filter 85 or a portion of the air-conducting line is located above the filter modules 10.1, 10.2, and 10.3, it is effectively prevented in a simple manner that the water from the water-conducting line 55a can rise up to the air filter 85 and, if applicable, impair its function.

The provision of an air filter 85 does not impair the method in any way.

The air 80 used is not particularly restricted; in particular, process air is used.

During the carrying out of step d), the total volume flow of air is additionally measured with a measuring apparatus (not shown) through the filter modules 10.1, 10.2, 10.3.

In step e) of the method according to the invention, which is carried out while steps d1) or d2) are still being continued, an automatic detection of a chain of air bubbles takes place if it occurs (symbolized by the dashed arrow 80, top left in FIG. 7). In addition, the volume flow is measured in steps d1) or d2) and thus also in step e), since step e) is carried out while steps d1) or d2) continue. The detection of an occurring chain of air bubbles is effected in FIG. 8 with the aid of a scattered light sensor 120, which measures the absorption. Simultaneously, the total volume flow is capture automatically in steps d1) or d2) and the simultaneous step e).

If the virus filtration modules 10.1, 10.2 and 10.3 are intact, i.e., they do not exhibit any damage, such as cracks, and therefore fulfill the desired filter function, the air 80 remains in the hollow fibers, since the air 80 cannot penetrate the pores of the hollow fiber wall. A chain of air bubbles is not detected by the scattered light sensor 120 in the line 75a, which is combined into one line, on the filtrate side 75.1, 75.2, 75.3. The volume flow of air 80 is at most 8 mL/min per virus filtration module 10.1, 10.2 and 10.3.

If one or more of the virus filtration modules 10.1, 10.2 and 10.3 are not intact, i.e., there are damages such as cracks and therefore will not fulfill the desired filter function, the air 80 passes through the damages in the hollow fiber wall and thus reaches the filtrate side 75.1, 75.2 and 75.3 of the filter module 10.1, 10.2 and 10.3, where a chain of air bubbles then occurs in the respective line 75.1a, 75.2a or 75.3a. At the same time, the measured volume flow of air 80 is above 8 mL/min for at least one of the virus filtration modules 10.1, 10.2 and 10.3. If only very minor damage is present, no chain of air bubbles is usually detected, but the measured volume flow of air 80 is then above 8 mL/min for at least one of the virus filtration modules 10.1, 10.2 and 10.3 and indicates a defect.

According to the embodiment shown, the lines 75.1a, 75.2a or 75.3a on the filtrate side 75.1, 75.2, 75.3 are combined to form a line 75a, on which the detection is carried out by optical means, in particular by a scattered light sensor 120. Moreover, for measuring the (total) volume flow of air, only one measuring apparatus (not shown) can be present for all virus filtration modules 10.1, 10.2 and 10.3. As a result, only one detection apparatus and only one measuring apparatus for the total volume flow of air are required, which significantly simplifies and accelerates the detection and evaluation of the test. If one or more of the virus filtration modules 10.1, 10.2 and 10.3 is/are not intact, a continuous chain of air bubbles is then detected in the sensor 120 for a duration of at least 20 seconds and the measured volume flow of the air 80 is above 8 mL/min for at least one of the virus filtration modules 10.1, 10.2 and 10.3.

According to the illustrated embodiment, the scattered light sensor 120 is arranged here on a part of the line 75a running from bottom to top, so that a chain of air bubbles in the line can be detected particularly well and completely as they rise.

Even if a virus filtration module 10.1, 10.2 and 10.3 is completely intact, a minimal loss of injected air 80 (the so-called leak flow) could nevertheless be observed in tests after passage through the filtration module 10.1, 10.2, 10.3. However, this does not play a role in carrying out the integrity test.

After passing through the filter modules 10.1, 10.2 and 10.3, the air 80 can be disposed of and collected, for example, in a waste collection container (not shown).

In the illustrated embodiment of FIG. 8, two sapphire glass windows 78.1 and 78.2 are installed for the measurement by optical means, which are arranged in the beam path of the sensor 120 in the line 75a.

It is expedient to provide in each case an additional outlet filter (not shown) at the filter outlet in the lines 75.1a, 75.2a and 75.3a on the filtrate side 75.1, 75.2 and 75.3, which removes potential contaminants, so that the scattered light sensor 120 does not capture absorption signals from undesired components and the measurement is not impaired.

The detection (step e)) by the scattered light sensor 120, the measurement of the volume flow of air and also the evaluation (step f)) are effected in a particularly fully automated manner, for example using a computer program in the form of a process control system (PCS) (not shown), in which the scattered light sensor 120 is integrated. In FIG. 8, the process control system is part of a method-related filtration apparatus, in which virus removal can be carried out. In the filtration apparatus shown in the section, the two integrity tests and the virus filtration are therefore carried out: An integrity test (pre-visual leak test) of the virus filtration modules 10.1, 10.2 and 10.3 is effected prior to virus filtration and an integrity test (post-visual leak test) of the virus filtration modules 10.1, 10.2 and 10.3 is effected after virus filtration, without it being necessary to install and remove the virus filtration modules 10.1, 10.2 and 10.3 for the tests. The functional testing of the filtration and the actual filtration therefore take place for the virus filtration modules 10.1, 10.2 and 10.3 in the filtration apparatus shown. As a result, the risk of potential damage or contamination of the modules is significantly reduced.

The embodiment illustrated in FIG. 8 has the advantage that the time required per test for each filter module can be significantly reduced. A plurality of filter modules can be tested simultaneously, wherein significant time savings, in particular with an automated test procedure, result. This can be particularly important for large-scale industrial applications.

If a defect is detected during simultaneous testing of virus filtration modules 10.1, 10.2 and 10.3, an individual testing of the virus filtration modules will be effected. For this purpose, the method steps are carried out only on one virus filtration module, e.g., virus filtration module 10.1, and the lines for the other virus filtration modules, e.g., 10.2 and 10.3, are kept closed. If the defective virus filtration module is identified, it is replaced and the method according to steps b) to e) is repeated with all virus filtration modules.

FIG. 9 illustrates a further embodiment of the present invention. If the same explanations apply as for FIG. 8, a corresponding repetition is omitted. In particular, as in FIG. 7, the water 70 is fed from below (from the feed side 55.1, 55.2 and 55.3 of the virus filtration modules 10.1, 10.2 and 10.3). In contrast to FIG. 8, however, in FIG. 9 the air 80 is not fed from below, but from above (from the retentate side 65.1, 65.2 and 65.3 of the virus filtration modules 10.1, 10.2 and 10.3). The terms “from below” or “from above” should be interpreted as broadly as possible, as has already been explained in detail.

In detail, in the embodiment illustrated in FIG. 9, after carrying out steps a), b), c) and, if applicable, the optionally carried out intermediate step and a leak test, which are effected as explained by way of example in FIG. 8, step d2) is carried out. In step d2), for each virus filtration module, the lines on the retentate side and the lines on the filtrate side are opened and the lines on the feed side are closed, wherein the pressure is maintained in order to feed air at a constant pressure at the same level as the water pressure from the lines on the retentate side (from above) into the virus filtration module. In detail, in FIG. 9, the lines 65.1a, 65.2a and 65.3a on the retentate side 65.1, 65.2 and 65.3 (at the upper end of the virus filtration modules 10.1, 10.2 and 10.3) and the lines 75.1a, 75.2a and 75.3a on the filtrate side 75.1, 75.2 and 75.3 of the respective filter modules 10.1, 10.2 and 10.3 are opened and the lines 55.1a, 55.2a and 55.3a on the feed side 55.1, 55.2 and 55.3 (at the lower end of the filter modules 10.1, 10.2 and 10.3) remain closed.

Opening the lines does not cause a pressure drop. Rather, the pressure remains constant throughout the test procedure at the pressure value used for the test procedure, which was selected from the range of at most 98±5 kPa.

The opening and closing of the lines can be effected by means of a device known to a person skilled in the art. Valves are shown in FIG. 9; other options are conceivable.

After opening the lines on the retentate side and the lines on the filtrate side, air 80 is fed via the valves 85v1 and 85v2 into the lines 65.1a, 65.2a and 65.3a, which are located in each case on the retentate side 65.1, 65.2 and 65.2 of the virus filtration modules 10.1, 10.2, 10.3, at a constant pressure of at most 98±5 kPa to the respective virus filtration modules 10.1, 10.2, 10.3. The air pressure is set to the same level as the previously set water pressure.

In FIG. 9, according to one embodiment—as in FIG. 7—the air 80 is initially passed through an air filter 85 in order to remove impurities from the air, so that they do not enter one of the filter modules 10.1. 10.2 and 10.3. The air filter 85 is arranged above the virus filtration modules 10.1, 10.2 and 10.3.

The volume flow of air 80 through the virus filtration modules 10.1, 10.2 and 10.3 is measured during step d2).

In step e) of the method according to the invention, an automatic detection of a chain of air bubbles is then effected if it occurs (symbolized by the dashed arrow 80, top left in FIG. 8). The detection is effected in FIG. 9 with the aid of a scattered light sensor 120, which measures the absorption. Further details are explained in FIG. 8. In particular, step e) takes place while step d2) continues to be carried out, so that simultaneously with the automatic detection of for the chain of air bubbles, the volume flow of the air 80 is also measured, as already explained in FIG. 8.

The present disclosure thus provides for the first time a partially or fully automated method or a corresponding filtration apparatus for an integrity test with which the test can be carried out reliably, reproducibly and in a simple manner.

In the following, the method of the disclosure is further illustrated with reference to examples without limiting the disclosure herein.

### EXEMPLARY EMBODIMENTS

The method of the present invention is explained in detail in the examples with reference to various embodiments.

### Example 1

### Visual Leak Test (VLT) for a Virus Filtration Module

### 1. Preparation of the Filtration Unit and Filter Modules

a. Initially, the required media connections were established to the filtration unit. These included, e.g., all hose connections for the required liquid feed and discharge lines. This also comprised the feed of process air and the provision of electricity, e.g., for measuring devices, sensors and the like for operating the filtration unit. The liquids used can be, for example, water or a buffer solution including water. The liquid used in Example 1 was an aqueous acetic acid/acetate buffer solution
b. All lines and line connections were initially subjected to hot steam sterilization at up to 130° C., since virus filtration is to be carried out following the visual leak test. For this purpose, a fitting piece for closing the open section was inserted into the filtration apparatus instead of a virus filtration module. The line system was then placed under overpressure to approximately 100 kPa in order to prevent the entry of contamination. Finally, a pressure reduction took place to a pressure P≤10 kPa, wherein a pressure equalization was effected throughout the system. At all points in the system that have an influence on the filter module, the pressure after completion of this process was therefore P≤10 kPa. This was effected to protect the virus filtration module from damage.
c. The installation of the filter module into the filtration unit was effected, wherein the hollow fibers runs vertically (the retentate side of the filter module was on top, the feed side was on the bottom, as shown in FIG. 3a). The number of filter modules is flexible depending on requirements. In the present Example 1, a virus filtration module manufactured by Asahi Kasei Medical CO., LTD., model PLANOVA 20N4, was installed.

### 2. Filling the Filtration Unit Including the Previously Installed Filter Module

a. Initially, a filling of the lines in the filtration unit was effected by means of executing an internal process control system (PCS) program without the installed filter module. For this purpose, the corresponding valves on the filter module were closed. This prevents possible damage to the modules. The selected pressure was ≤98±5 kPa. It was then checked that there were no more air bubbles in the outflowing liquid, since the line system was then completely filled. This can be effected visually by an employee or with a sensor. In the present Example 1, this was undertaken visually.
b. The filling of the filter module was started, wherein initially only the inner region of the hollow fibers, i.e., the “hollow fiber interior,” was filled from the feed side (from below). In FIG. 1, this corresponds to the feed of liquid starting from A to B. According to FIG. 3a, liquid was therefore passed via the inlet at 55 of the filter module to the outlet at 65. The outlets at 75o+75u were in each case closed with valves. The aim was to completely fill the “hollow fiber interior.” As a parameter for checking the complete filling, the differential pressure across the filter module was determined at 98 kPa±5 kPa. The differential pressure was ascertained by two pressure measuring devices, one before and one after the filter module, wherein the difference between the two pressures was measured. If the pressure before and after the filter module is approximately the same, the filter module is completely filled. An additional parameter was the reaching of the dead volume. The dead volume is considered to be the volume of the entire line system including all filter modules used (here: one filter module). This depends on the particular system, the type and number of filter modules and can easily be ascertained by a person skilled in the art in each individual case or can be requested from the manufacturer. The 4 m² filter module used had a dead volume of 1,900 mL according to the manufacturer's specifications.
c. The filling of liquid into the filter module, namely the interstices between the hollow fibers, was then started from the feed side (from below). This corresponds in FIG. 1 from A through the filter module to C. According to FIG. 3a, liquid was therefore passed from the inlet at 55 of the filter module to the outlet at 75o of the filter module. The outlet at 65 (and 75u) remained closed. The aim was to fill the entire line system and the filter module with liquid as completely as possible. The differential pressure across the filter module at 98 kPa±5 kPa and the reaching of the dead volume were again used as parameters for checking the complete filling of the interstices in the filter module.

### 3. Optional Emptying of the Inner Region of the Filter Module

For this purpose, the liquid in the inner region of the hollow fibers was removed again. For this purpose, the valve 55v was opened by means of the PCS program as shown in FIG. 6b, so that the buffer solution could flow out of the filter module via the line 55a until the inner region of the hollow fibers and also the connected line 55a had been completely emptied. In addition, according to FIG. 6b, the inlet at the line 65a was opened and the filter module was ventilated via the path 85a by opening the valve 85v via the air filter 85. The parameter is that no more liquid exits the outlet of the filtration unit. This can be effected by trained employees through visual inspection or via a sensor with the aid of the PCS program. In this example, this was carried out visually.

This optional step is not necessary in every case. This step has the advantage that the internal emptying of the virus filtration module represents a particularly gentle procedure for the subsequent visual leak test. If the inner region of the hollow fibers is filled with liquid and then exposed to gas for the visual leak test, this could cause additional expansion of the hollow fibers, as a result of which the stress on the filter module is further increased.

### 4. Visual Leak Test (VLT)

a. Leak test of the filtration system: prior to the start of the visual leak test, initially a leak test of the filtration system and the installed filter module can be effected to rule out any leaks in the system, e.g., due to an improperly installed seal. Although this is not necessary due to the measurement of the volume flow of the process air, it is described here for the sake of completeness, in order to demonstrate the procedure with reference to an example. The leak test was carried out by means of a PLC program. A PLC program is expedient since, in addition to a continuous chain of air bubbles, the scattered light measurement also allows the total volume flow of process air through the filtration system to be reliably measured during the visual leak test. The leak test (also known as pressure hold test) serves to ensure that no air escapes from the system, as a result of which the subsequent integrity test and its evaluation could be impaired.

According to part 1 of the PLC program, valves 85.1v, 85.2v, 65.1v and 75.1v were opened analogously to FIG. 9, but only for one virus filtration module (module 10.1). The valve 55.1v remained closed, since the air is to be injected from the retentate side (from above). The inlets and outlets not required on the 55v and 75v valves of the system were closed. A further outlet on the filter module not shown in FIG. 9 (see FIG. 3a: outlet at 75u) was also closed on the filter module. In the present example, the process air was therefore fed into the virus filtration module from the retentate side (from above). A step-by-step pressure buildup was effected by means of a controlled pressure increase (a so-called fixed ramp), which was achieved using process air until the test pressure (also referred to as pre-stabilization pressure) was reached. In this example, the pressure in the system was increased from 0 kPa to 70 kPa. The pre-stabilization pressure was therefore 70 kPa. A stabilization period of 180 s was selected. The stabilization period was the time during which the process air was able to disperse in the system before the leak test was evaluated. The leak test is considered passed if the pressure drop after the stabilization period is ≤10 kPa, in particular ≤5 kPa.

After the optional leak test, the PLC program evaluated whether or not the pressure drop during this period (≤10 kPa) was maintained:
Pressure drop: ≤10 kPa:
   If the pressure drop is below the permitted maximum pressure loss, it must be assumed that there is no leak in the filtration system. In this case, the PLC program continues according to item 4b.
Pressure drop >10 Kpa
   If the pressure drop is above the permitted maximum pressure loss, it must be assumed that there is a leak in the system. In this case, the PLC program is terminated and the leak in the filtration system is searched for and eliminated and the PLC program is started again at item 4a by means of repeating it.

In the present Example 1, the pressure drop was ≤10 kPa, and the leak test of the filtration system was therefore considered passed. The visual leak test according to item 4b could be carried out.

b. Carrying out of the visual leak test: According to Part 2 of the Plc Program, analogous to FIG. 9, but using only one filtration module (here: module 10.1), in addition to the already opened valves 85.1v, 85.2v, 65.1v, 75.1v, the outlet at valve 75v of the filtration system was opened. The inlets and outlets not required (here: at the valve 55v) in the filtration system and valve 55.1v on the filter module were closed. A filter module outlet not shown in FIG. 9 for reasons of clarity (see, e.g., the outlet at 75u in FIG. 3a) was also closed. The process air was fed to the virus filtration module from the retentate side (from above). The existing pressure was further increased with process air (so-called continuation of the ramp) until the final test pressure of 98±5 kPa was reached. After a stabilization period, a continuous evaluation was effected during the test period, wherein
the detection was carried out by a visible chain of air bubbles and
the volume flow of process air flowing into the system was measured.

The parameters for part 2 of the PLC program were as follows:

The stabilization period was 600 s: This was the time during which the process air could further be dispersed in the system before the start of the evaluation was effected in the PLC program. The start of the evaluation corresponded to the end of reaching the test pressure.

The test pressure was 98±5 kPa: this was the pressure at which the filtration system was subjected to process air during the test period.

The test period was 300 s: This was the period during which the test pressure was constant and the continuous control for the presence of process air was detected at the scattered light sensor (see FIG. 7). In addition, the amount of process air, or more precisely the volume flow of the method air flowing into the system, was continuously captured by the PLC.

The measured maximum permitted volume flow Volₘₐₓ of air can be up to 8 mL/min/filter module: This represents the maximum permitted volume flow of air during the test period. It is an expected value for the maximum volume flow of air for an intact filter module. Experience has shown that the volume flow of air is between 0.8-8 mL/min/filter module. It must be noted that when testing a plurality of filter modules, the volume flows are cumulative. For example, the maximum permitted volume flow is Volₘₐₓ of the air with seven filter modules =7*8 mL/min≤56 mL/min. If only one hollow fiber is defective in the tested filtration module, a volume flow of air of >150 mL/min is measured.

After the completion of the visual leak test, evaluation with a computer program in the form of a PLC was effected as to whether the scattered light detector of the scattered light measurement, in cooperation with the PLC program, produced a continuous chain of air bubbles ≥20 s during the entire test period. In addition, it was checked whether the maximum permitted volume flow Volₘₐₓ of the air was maintained throughout the entire test period. Briefly occurring air bubbles are to be disregarded, so long as they do not occur as a chain of air bubbles lasting ≥20 s.

Therefore, the following three cases are distinguished when testing one virus filtration module:

### Case 4.b.1:

Volₘₐₓ is above 8 mL/min+chain of air bubbles ≥20 s or
Volₘₐₓ is at most 8 mL/min+chain of air bubbles ≥20 s or
Volₘₐₓ is above 8 mL/min+chain of air bubbles ≤20 s.

Note: It is assumed that the case “Volₘₐₓ is at most 8 mL/min+chain of air bubbles ≥20 s” cannot occur. This would mean that a medium to large defect would be present (chain of air bubbles ≥20 s), but would not be recognized by the measurement of volume flow, which captures the smallest defects. However, as a precaution, all (theoretically conceivable) variants are always checked during testing. This case has no practical significance.

### Case 4.b.2:

Volₘₐₓ is at most 8 mL/min+chain of air bubbles ≤20 s.

For case 4.b.1, the following applies in all three cases: It must be assumed that the filter module is defective. In this case, the method continues with item 5.a.

For case 4.b.2, the following applies: There is no defect in the filter module. In this case, the method continues with item 5.b.

In the present Example 1, the virus filtration module tested was a Volₘₐₓ measured below 8 mL/min and no chain of air bubbles was detected. The filter module tested had no defects. It was possible to proceed directly to item 5.b.

For the sake of completeness, it will also be explained how to proceed if a filter module is defective:

### 5. Evaluation of the Visual Leak Test

The computer program in the form of the PLC program decides in an automated manner how to proceed without the possibility of influence by the employees.
a. Case 4.b.1 (defective filter module(s))
i. Pre-visual leak test (prior to virus filtration):
   When testing a plurality of filter modules simultaneously in which a defect has been detected, individual testing of the filter modules is carried out, wherein the above steps 2 to 4 are repeated. The individual testing in a setup as in FIG. 8 or 9 is effected so that the lines of the non-tested filter modules remain permanently closed during the tests.
a. The detection and replacement of the defective filter module(s) are effected, and steps starting with 1.c. to 4. are repeated either with the replaced filter module only or with all filter modules until all filter modules have passed the visual leak test.
b. Now that all filter modules are intact, virus filtration of the product is carried out.
ii. Post-visual leak test (after virus filtration):
   The filter modules are tested again individually, repeating steps 2 to 4. The individual testing in a setup as in FIG. 8 or 9 is effected so that the lines of the non-tested filter modules remain permanently closed during the tests.
a. The detection and thus verification of the result from item 4 is effected.
b. If the result again confirms a defect in one or more filter modules, the procedure described in the following item i. is followed.
i. Since a defect in one or more filter modules is assumed, it must be assumed that the potentially present viruses were not successfully separated and the entire virus filtration method step must be repeated. The procedure continues with item 5.a.
b. Case 4.b.2 (no defective filter module)
i. Pre-visual leak test (prior to virus filtration):
   There is no defect in the tested filter modules. The pre-visual leak test has been passed. Virus filtration of the product can be carried out immediately.
ii. Post-visual leak test (after virus filtration):
   The visual leak test is continued according to item 4.b. If no defect is found in the tested filter modules, the post-visual leak test is passed. The subsequent gold particle test (GPT test) can then be carried out immediately. According to the manufacturer Asahi Kasei, this GPT test is necessary as an additional test for the release of the product for further processing.

According to the present Example 1, it was possible to proceed according to case 4.b.2. The pre-visual leak test was passed. The virus filtration of the product could be connected immediately. The following post-visual leak test was also passed. Thereafter, the gold particle test (GPT test) was carried out and passed. The filtered and now virus-free product could be released.

### Example 2

### Visual Leak Test for a Virus Filtration Module

A virus filtration module from Asahi Kasei Medical CO., LTD., model PLANOVA 20N 4, was provided. The procedure was as in Example 1, but in items 2.b and 2.c the liquid was fed into the filtration module from the retentate side (from above).

A leak test was not necessary, since the volume flow of air was monitored throughout the test period. In the visual leak test, a Volₘₐₓ below 8 mL/min was measured for the tested virus filtration module, and no chain of air bubbles was detected. The filter module tested had no defects. It was possible to proceed directly to item 5.b. Therefore, the pre-visual leak test was passed. The virus filtration of the product could be connected immediately. The following post-visual leak test was also passed. Thereafter, the gold particle test (GPT test) was successfully carried out. The filtered and now virus-free product could be released.

### Example 3

### Visual Leak Test For a Virus Filtration Module

A virus filtration module from Asahi Kasei Medical CO., LTD., model PLANOVA 20N 4, was provided. The procedure was as in Example 1, but in item 4.b. the process air was fed to the virus filtration module from the feed side (from below).

A leak test was not necessary, since the volume flow of air was monitored throughout the test period. In the visual leak test, a Volₘₐₓ below 8 mL/min was measured for the tested virus filtration module, and no chain of air bubbles was detected. The filter module tested had no defects. It was possible to proceed directly to item 5.b. Therefore, the pre-visual leak test was passed. The virus filtration of the product could be connected immediately. The following post-visual leak test was also passed. Thereafter, the gold particle test (GPT test) was successfully carried out. The filtered and now virus-free product could be released.

### Examples 4 To 6

### Visual Leak Test for a Virus Filtration Module

Examples 1 to 3 were in each case repeated using a virus filtration module from Asahi Kasei Medical CO., LTD., model PLANOVA 20N4, wherein, however, optional step 3 (emptying the inner region of the virus filtration module) was omitted. An aqueous acetic acid/acetate buffer was used as buffer solution.

In all Examples 4 to 6, the leak test was omitted since the volume flow of air was monitored throughout the test period. In the visual leak test, a Volₘₐₓ below 8 mL/min was measured for each tested virus filtration module, and no chain of air bubbles was detected. The filter modules tested had no defects. It was possible to proceed directly to item 5.b. Therefore, the pre-visual leak test was passed. Virus filtration of the product could be carried out immediately. The following post-visual leak test was also passed. Thereafter, the gold particle test (GPT test) was successfully carried out. The filtered and now virus-free product could be released.

### Example 7

### Visual Leak Test for Three Virus Filtration Modules

Three virus filtration modules from Asahi Kasei Medical CO., LTD., model PLANOVA 20N4, were provided, which were to be simultaneous tested. The procedure was analogous to Example 1.

The leak test ran successfully. A defect was found in the visual leak test: Volₘₐₓ was >150 mL/min and a chain of air bubbles ≥20 s was detected. At least one of the filter modules tested was defective. Since a chain of air bubbles occurred for ≥20 s, a medium to large defect could be assumed.

The procedure was as in case 4.b.1 and an individual test was carried out with each of the 3 filter modules in the pre-visual leak test (prior to virus filtration). At the 3rd filter module, the defect was detected, and it was replaced. Then, steps 2 to 4 were repeated and carried out with all three filter modules simultaneously. The filter modules passed the pre-visual leak test. The product could be filtered.

For clear verification, a new individual test of the filter modules was carried out in the post-visual leak test (after virus filtration), repeating steps 2 to 4. The post-test confirmed the results.

Subsequently, the gold particle test (GPT test) was successfully carried out. The filtered and now virus-free product could be released.

### Example 8

### Visual Leak Test for Seven Virus Filtration Modules

Seven virus filtration modules from Asahi Kasei Medical CO., LTD., model PLANOVA 20N4, were provided in order to simultaneously test them. The procedure was as in Example 1 but omitting the optional step 3 and the leak test.

In the visual leak test, the virus filtration modules tested showed a Volₘₐₓbelow 56 mL/min measured (7×8 mL/min) and no chain of air bubbles was detected. The seven filter modules tested simultaneously had no defects. It was possible to proceed directly to item 5.b. Therefore, the pre-visual leak test was passed. Virus filtration of the product could be carried out immediately. The following post-visual leak test was also passed. Thereafter, the gold particle test (GPT test) was successfully carried out. The filtered and now virus-free product could be released.

### Example 9

### Visual Leak Test for Seven Virus Filtration Modules

Seven virus filtration modules from Asahi Kasei Medical CO., LTD., model PLANOVA 20N4, were provided in order to simultaneously test them. The procedure was as in Example 3, i.e., the process air in item 4.b. was fed to the virus filtration module from the feed side (from below).

In the visual leak test, a Volₘₐₓ below 56 mL/min was measured for the tested virus filtration modules, and no chain of air bubbles was detected. The filter modules tested had no defects. It was possible to proceed directly to item 5.b. Therefore, the pre-visual leak test was passed. Virus filtration of the product could be carried out immediately. The following post-visual leak test was also passed. Thereafter, the gold particle test (GPT test) was successfully carried out. The filtered and now virus-free product could be released.

### LIST OF REFERENCE SIGNS:

10, 10.1, 10.2, 10.3 Virus filtration module, hollow fiber filter module
20 Lower end of a hollow fiber
30 Upper end of a hollow fiber
35 Inner region of a hollow fiber
38.1, 38.2 Interstices between hollow fibers
40 Wall of a hollow fiber
45 Pore
50 Bundles of hollow fibers
50.1. 50.2, 50.1a, 50.1b Hollow fibers
55, 55.1, 55.2, 55.3 Feed side
65, 65.1, 65.2, 65.3 Retentate side
75, 750, 75u, 75.1, 75.2,
75.3 Filtrate side
78.1, 78.2 Transparent window
55a, 55.1a, 55.2a, 55.3a Line on the feed side
65a, 65.1a, 65.2a, 65.3a Line on the retentate side
70 Liquid, water
75a, 75b, 75.1a, 75.2a, 75.3a Line on the filtrate side
55v, 55.1v, 55.2v, 55.3v, 55v Valves in the line on the feed side
65v, 65.1v, 65.2v, 65.3v Valves in the line on the retentate side
75v, 75.1v, 75.2v, 75.3v,
75av, 75bv Valves in the line on the filtrate side
77.1, 77.2 Openings
78.1, 78.2 Windows
79.1. 79.2, 79.3, 79.4 Seals
80 Air
75v, 85.1v, 85.2v Valves in air-conducting lines
85 Air filter
90 Damage
100 Filtration apparatus for carrying out the integrity test and virus filtration
110.1, 110.2 Viruses
120 Scattered light sensor
120.1 Light source
120.2, 120.3, 120.4 Detectors
130 Chain of air bubbles
L Light
L1, L2, L3 Light beams
H Horizontal axis
V Vertical axis

The invention comprises aspects disclosed in the following clauses:

### CLAUSES

1. A method for testing the integrity of a virus filtration module (10, 10.1, 10.2, 10.3), comprising the steps of:
   a) installing a virus filtration module (10, 10.1, 10.2, 10.3) in a filtration apparatus (100), wherein the virus filtration module (10, 10.1, 10.2, 10.3) is in the form of a hollow fiber filter module comprising hollow fibers (50.1, 50.2, 50.1a, 50.1b) having an inner region (35) and hollow fiber interstices (38.1, 38.2), which has a feed side (55, 55.1, 55.2, 55.3), a retentate side (65, 65.1, 65.2, 65.3) and a filtrate side (75, 75.1, 75.2, 75.3) and in each case has a line (55a, 55.1a, 55.2a, 55.3a) on the feed side (55, 55.1, 55.2, 55.3), a line (65a, 65.1a, 65.2a, 65.3a) on the retentate side (65, 65.1, 65.2, 65.3) and a line (75a, 75.1a, 75.2a, 75.3a) on the filtrate side (75, 75.1, 75.2, 75.3);
   b) filling the virus filtration module (10, 10.1, 10.2, 10.3) and the connected lines with liquid (70), at a constant pressure of at most 98±5 kPa with the following steps: b1) filling all lines connected to the virus filtration module (10, 10.1, 10.2, 10.3) with liquid (70), but without filling the virus filtration module (10, 10.1, 10.2, 10.3); b2) filling the inner region (35) of the hollow fibers (50.1, 50.2, 50.1a, 50.1b) of the virus filtration module (10, 10.1, 10.2, 10.3) with liquid (70), wherein the line (55a, 55.1a, 55.2a, 55.3a) on the feed side (55, 55.1, 55.2, 55.3) and the line (65a, 65.1a, 65.2a, 65.3a) on the retentate side (65, 65.1, 65.2, 65.3) are opened and the line (75a, 75.1a, 75.2a, 75.3a) on the filtrate side (75, 75.1, 75.2, 75.3) is closed and b 3) filling the interstices (38.1, 38.2) of the hollow fibers (50.1, 50.2, 50.1a, 50.1b) of the virus filtration module (10, 10.1, 10.2, 10.3) with liquid (70), wherein either the line (55a, 55.1a, 55.2a, 55.3a) on the feed side (55, 55.1, 55.2, 55.3) and the line (75a, 75.1a, 75.2a, 75.3a) on the filtrate side (75, 75.1, 75.2, 75.3) are opened and the line (65a, 65.1a, 65.2a, 65.3a) on the retentate side (65, 65.1, 65.2, 65.3) are closed or the line (65a, 65.1a, 65.2a, 65.3a) on the retentate side (65, 65.1, 65.2, 65.3) and the line (75a, 75.1a, 75.2a, 75.3a) on the filtrate side (75, 75.1, 75.2, 75.3) are opened and the line (55a, 55.1a, 55.2a, 55.3a) on the feed side (55, 55.1, 55.2, 55.3) is closed; wherein in steps b2) and b3) the liquid (70) is fed to the virus filtration module (10, 10.1, 10.2, 10.3) from the line (55a, 55.1a, 55.2a, 55.3a) on the feed side (55, 55.1, 55.2, 55.3) or from the line (65a, 65.1a, 65.2a, 65.3a) on the retentate side (65, 65.1, 65.2, 65.3);
   c) stopping the feed of liquid (70) as soon as the virus filtration module (10, 10.1, 10.2, 10.3) and the lines are filled with liquid (70), wherein the pressure is maintained, and closing all lines;
   d) feeding air (80) to the virus filtration module (10, 10.1, 10.2, 10.3) with step d1) or d2):
d1) opening the line (55a, 55.1a, 55.2a, 55.3a) on the feed side (55, 55.1, 55.2, 55.3) and the line (75a, 75.1a, 75.2a, 75.3a) on the filtrate side (75, 75.1, 75.2, 75.3) of the virus filtration module (10, 10.1, 10.2, 10.3) and leaving the line (65a, 65.1a, 65.2a, 65.3a) on the retentate side (65, 65.1, 65.2, 65.3) closed, wherein the pressure is maintained in order to feed air (80) with a constant pressure at the same level as the liquid pressure from the line (55a, 55.1a, 55.2a, 55.3a) on the feed side (55, 55.1, 55.2, 55.3) into the virus filtration module (10, 10.1, 10.2, 10.3);
   or
d2) opening the line (65a, 65.1a, 65.2a, 65.3a) on the retentate side (65, 65.1, 65.2, 65.3) and the line (75a, 75.1a, 75.2a, 75.3a) on the filtrate side (75, 75.1, 75.2, 75.3) of the virus filtration module (10, 10.1, 10.2, 10.3) and leaving the line (55a, 55.1a, 55.2a, 55.3a) on the feed side (55, 55.1, 55.2, 55.3) closed, wherein the pressure is maintained in order to feed air (80) with a constant pressure at the same level as the liquid pressure from the line (65a, 65.1a, 65.2a, 65.3a) on the retentate side (65, 65.1, 65.2, 65.3) into the virus filtration module (10, 10.1, 10.2, 10.3);
   and
e) automatically detecting air bubbles (130) occurring in the line (75a, 75.1a, 75.2a, 75.3a) on the filtrate side (75, 75.1, 75.2, 75.3) of the virus filtration module (10, 10.1, 10.2, 10.3).
2. The method according to clause 1,
   characterized in that
the automatic detection of occurring air bubbles (130) in step e) is carried out by optical means, in particular a scattered light sensor (120) is used, wherein the line (75a, 75.1a, 75.2a, 75.3a) on the filtrate side (75, 75.1, 75.2, 75.3) for optical detection is selected so that it consists of or comprises transparent material.
3. The method according to clause 1,
   characterized in that
after carrying out step c), the liquid (70) present is completely emptied from the inner region (35) of the hollow fibers (50.1, 50.2, 50.1a, 50.1b) and the connected line (55a, 55.1a, 55.2a, 55.3a).
4. The method according to one of the clauses 1 to 3,
   characterized in that
in a step f) following step e), an evaluation, in particular an automatic evaluation, of step e) is carried out, wherein the test is evaluated as passed if during the test no continuous chain of air bubbles is detected for a duration of at least 20 seconds in the line (75a, 75.1a, 75.2a, 75.3a) on the filtrate side (75, 75.1, 75.2, 75.3).
5. The method according to one of the preceding clauses 1 to 4,
   characterized in that
the liquid (70) according to step b2) and b3) is fed to the virus filtration module (10, 10.1, 10.2, 10.3) from the line (55a, 55.1a, 55.2a, 55.3a) on the feed side (55, 55.1, 55.2, 55.3) and the air (80) according to step d2) is fed to the virus filtration module (10, 10.1, 10.2, 10.3) from the line (65a, 65.1a, 65.2a, 65.3a) on the retentate side (65, 65.1, 65.2, 65.3).
6. The method according to one of the preceding clauses 1 to 5,
   characterized in that
in steps b 2) and b 3), the filling level of the virus filtration module (10, 10.1, 10.2, 10.3) is monitored by:
   measuring the pressure prior to and after the virus filtration module (10, 10.1, 10.2, 10.3) and determining the differential pressure and/or
   determining the amount of liquid (70) fed in relation to the dead volume of the filtration apparatus (100), including the virus filtration module (10, 10.1, 10.2, 10.3), wherein: the amount of liquid (70) fed ≥ dead volume.
7. The method according to one of the preceding clauses 1 to 6,
   characterized in that
steps b) to e) and optionally f) are carried out with the support of a process control system, wherein in particular the process control system is selected so that it can also support the carrying out of virus filtration.
8. The method according to one of the preceding clauses 1 to 7,
   characterized in that
the method for testing the integrity of a virus filtration module (10, 10.1, 10.2, 10.3) according to steps a) to e) and optionally f) is carried out twice and between the two methods a virus filtration is effected with the tested virus filtration module (10, 10.1, 10.2, 10.3).
9. The method according to one of the preceding clauses 1 to 8,
   characterized in that
two, three, four, five, six, seven or more virus filtration modules (10, 10.1, 10.2, 10.3) are tested simultaneously in the method according to steps a) to e) and optionally f).
10. The method according to one of the preceding clauses 1 to 9,
   characterized in that
one, two, three or more of the following method steps are carried out:
   the liquid (70) is selected from water or a buffer solution including water;
   the water of the liquid (70) is selected from distilled water, bidistilled or deionized water, ultrapure water or sterile water, in particular water for injection purposes;
   the virus filtration module (10, 10.1, 10.2, 10.3) is arranged during the method so that the hollow fibers (50.1, 50.2, 50.1a, 50.1b) run in a horizontal direction or in a vertical direction, in particular in a vertical direction;
   prior to the entry of the liquid (70) into the virus filtration module (10, 10.1, 10.2, 10.3) in steps b1) and b2), the liquid (70) is passed through an inlet filter in order to filter out possible impurities and contaminations from the liquid (70);
   after the exit of the liquid (70) from the virus filtration module (10, 10.1, 10.2, 10.3) in steps b2) and b3), the liquid (70) is passed through an outlet filter;
   the air (80) is selected from process air;
   the air (80) is passed through an air filter (85) prior to the entry of the virus filtration module (10, 10.1, 10.2, 10.3);
   after step d) and prior to step e), a stabilization period of up to 600 s is observed, during which the air (80) is further dispersed;
   a counting apparatus for the air bubbles is used;
   transparent portions in the form of windows (78.1, 78.2) are provided in the line (75a, 75.1a, 75.2a, 75.3a) on the filtrate side (75, 75.1, 75.2, 75.3) for detection by optical means;
   windows (78.1, 78.2) made of transparent plastics material, selected from polystyrene (PS), polymethyl methacrylate (PMMA) or polycarbonate (PC), polyvinyl chloride (PVC), polyphenylene ether (PPO) or polyethylene (PE) or mixtures thereof; or selected from glass of hydrolytic class 1 or sapphire glass;
   in the case of detection by optical means, the detection apparatus (120) is arranged on a line (75a) running from bottom to top, so that the air bubbles (130) to be detected can rise in the line (75a);
   during step d1) or d2), the volume flow of the air (80) is measured;
   an air-conducting line (85a) leading from the air filter (85) is provided with a portion that has the highest point of the filtration apparatus (100), so that no liquid (70) can rise to the air filter (85);
   when testing a plurality of virus filtration modules (10.1, 10.2, 10.3) simultaneously, they are aligned parallel to one another with the hollow fibers (50.1, 50.2, 50.1a, 50.1b) running in the vertical direction;
   when testing a plurality of virus filtration modules (10.1, 10.2, 10.3) simultaneously, the liquid (70) is fed in each case via a line (55.1a, 55.2a, 55.3a or 65.1a, 65.2a, 65.3a) assigned to each virus filtration module (10.1, 10.2, 10.3) and from the same side for all virus filtration modules (10.1, 10.2, 10.3) to be tested;
   when testing a plurality of virus filtration modules (10.1, 10.2, 10.3) simultaneously, the air (80) is fed in each case via a line (55.1a, 55.2a, 55.3a or 65.1a, 65.2a, 65.3a) assigned to each virus filtration module (10.1, 10.2, 10.3) and from the same side for all virus filtration modules (10.1, 10.2, 10.3) to be tested;
   when testing a plurality of virus filtration modules (10.1, 10.2, 10.3) simultaneously, the respective lines (75.1a, 75.2a, 75.3a) on the filtrate side (75.1, 75.2, 75.3) are combined to form one line (75a), and the detection apparatus (120) is arranged on the combined line (75a);
   prior to carrying out steps a) to e), sterilization, in particular by hot steam sterilization, of the lines is carried out;
after sterilization of the lines and prior to carrying out step a), the lines are placed under an overpressure to approximately 100 kPa and subsequently a pressure reduction to a pressure P≤10 kPa in all lines is undertaken;
   prior to carrying out step d), a leak test is carried out on the filtration system (100) and the installed virus filtration module(s) (10.1, 10.2, 10.3);
and/or
   the filtration apparatus is designed so that virus filtration can be carried out.
11. The method according to one of the preceding clauses 1 to 10,
   characterized in that
if a defect is detected in the integrity test during simultaneous testing of a plurality of virus filtration modules (10.1, 10.2, 10.3), an individual test of the virus filtration modules (10.1, 10.2, 10.3) is carried out according to steps b) to e) and optionally f), the defective virus filtration module(s) (10.1, 10.2, 10.3) is/are replaced and the method according to steps b) to e) and optionally f) is repeated with the replaced virus filtration module(s) or with all virus filtration modules (10.1, 10.2, 10.3).
12. A filtration apparatus (100) for carrying out the method according to one of the clauses 1 to 11, comprising
   one, two, three, four, five, six, seven or more virus filtration modules (10, 10.1, 10.2, 10.3) which are in the form of hollow fiber filter modules, in each case comprising hollow fibers (50.1, 50.2, 50.1a, 50.1b) having an inner region (35) and hollow fiber interstices (38.1, 38.2), in each case having a feed side (55, 55.1, 55.2, 55.3), a retentate side (65, 65.1, 65.2, 65.3) and a filtrate side (75, 75.1, 75.2, 75.3);
   devices for attaching and fastening one or more virus filtration modules (10, 10.1, 10.2, 10.3);
   in each case, one line (55a, 55.1a, 55.2a, 55.3a) on the feed side (55, 55.1, 55.2, 55.3), one line (65a, 65.1a, 65.2a, 65.3a) on the retentate side (65, 65.1, 65.2, 65.3) and one line (75a, 75.1a, 75.2a, 75.3a) on the filtrate side (75, 75.1, 75.2, 75.3) of each virus filtration module (10, 10.1, 10.2, 10.3) for feeding liquid (70) and/or air (80) at a constant pressure of at most 98±5 kPa to the virus filtration module(s) (10, 10.1, 10.2, 10.3);
   opening and closing devices (55v, 55.1v, 55.2v, 55.3v, 65v, 65.1v, 65.2 65.3v, 75v, 75.1v, 75.2v, 75.3v, 75av, 75bv, 85v, 85.1v, 85.2v) for the respective lines;
   a connector for feeding liquid (70) at a constant pressure of at most 98±5 kPa having the lines to the virus filtration module(s) (10, 10.1, 10.2, 10.3);
   a connector for feeding air (80) of a constant pressure at the same level as the liquid pressure having the lines to the virus filtration module(s) (10, 10.1, 10.2, 10.3); and
   a detection apparatus (120) for automatically detecting air bubbles (130) in the line (75a) on the filtrate side (75, 75.1, 75.2, 75.3) of the virus filtration module(s) (10, 10.1, 10.2, 10.3).
13. The filtration apparatus (100) according to clause 12,
   characterized in that
the detection apparatus represents a scattered light sensor (120), wherein the line (75a, 75.1a, 75.2a, 75.3a) on the filtrate side (75, 75.1, 75.2, 75.3) for optical detection is selected so that it consists of or comprises transparent material.
14. The filtration apparatus (100) according to clause 12 or 13,
   characterized in that
it has one, two, three, four or more of the following features:
   transparent portions in the form of windows (78.1, 78.2) are provided in the line (75a, 75.1a, 75.2a, 75.3a) on the filtrate side (75, 75.1, 75.2, 75.3) for detection by optical means;
   transparent portions in the form of windows (78.1, 78.2) made of transparent plastics material, selected from polystyrene (PS), polymethyl methacrylate (PMMA) or polycarbonate (PC), polyvinyl chloride (PVC), polyphenylene ether (PPO) or polyethylene (PE) or mixtures thereof; or selected from glass of hydrolytic class 1 or sapphire glass;
   the virus filtration module (10, 10.1, 10.2, 10.3) is arranged in the filtration apparatus so that the hollow fibers in the virus filtration module (10, 10.1, 10.2, 10.3) run in a horizontal direction or in a vertical direction, in particular in a vertical direction;
   the opening and closing devices for the lines are selected from valves;
   in the case of detection by optical means, the detection apparatus (120) is arranged on a line (75a) running from bottom to top, so that the air bubbles (130) can rise in the line (75a);
   a measuring apparatus is provided for measuring the volume flow of the air (80) through the virus filtration module (10, 10.1, 10.2, 10.3);
   an air-conducting line (85a) leading from the air filter (85) has a portion that represents the highest point of the filtration apparatus (100) so that no liquid (70) can rise to the air filter (85);
   if a plurality of virus filtration modules (10.1, 10.2, 10.3) are present in the filtration apparatus (100) simultaneously, they are in each case aligned parallel to one another with the hollow fibers (50.1, 50.2, 50.1a, 50.1b) running in the vertical direction;
and/or
   if a plurality of virus filtration modules (10.1, 10.2, 10.3) are present in the filtration apparatus (100) simultaneously, the respective lines (75.1a, 75.2a, 75.3a) on the filtrate side (75.1, 75.2, 75.3) are combined to form a line (75a), and the detection apparatus (120) is arranged on the combined line (75a).
15. The filtration apparatus (100) according to one of clauses 12 to 14,
   characterized in that
the filtration apparatus (100) for carrying out the method for testing the integrity of a virus filtration module (10, 10.1, 10.2, 10.3) according to one of clauses 1 to 12 is designed so that virus filtration can be carried out therein with the virus filtration module(s) (10, 10.1, 10.2, 10.3) being tested.
16. A method for cultivating prokaryotic or eukaryotic cells in liquid cell culture in a bioreactor to produce a product, in particular a protein, and carrying out virus filtration of the product obtained using one or more virus filtration modules (10, 10.1, 10.2, 10.3), wherein the method for testing the integrity of the one or more virus filtration modules (10, 10.1, 10.2, 10.3) according to one of the preceding clauses 1 to 11 is carried out prior to and/or after virus filtration.
17. A method for producing a recombinant protein, wherein the method comprises the following steps:
step a) cultivating prokaryotic or eukaryotic cells expressing a recombinant protein in cell culture in a bioreactor;
step b) harvesting the recombinant protein;
step c) purifying the recombinant protein;
step d) removing viruses from the recombinant protein obtained in a virus filtration with one or more virus filtration modules (10, 10.1, 10.2, 10.3), wherein the method for testing the integrity of the one or more virus filtration modules (10, 10.1, 10.2, 10.3) according to one of the preceding clauses 1 to 11 is carried out prior to and/or after virus filtration.

1. A method for testing the integrity of one or more virus filtration modules (10, 10.1, 10.2, 10.3), comprising the steps of:
a) installing one or more virus filtration modules (10, 10.1, 10.2, 10.3) in a filtration apparatus (100), wherein each virus filtration module (10, 10.1, 10.2, 10.3) is in the form of a hollow fiber filter module, in each case comprising hollow fibers (50.1, 50.2, 50.1a, 50.1b) having an inner region (35) and hollow fiber interstices (38.1, 38.2), a feed side (55, 55.1, 55.2, 55.3), a retentate side (65, 65.1, 65.2, 65.3) and a filtrate side (75, 75.1, 75.2, 75.3) and in each case having a line (55a, 55.1a, 55.2a, 55.3a) on the feed side (55, 55.1, 55.2, 55.3), a line (65a, 65.1a, 65.2a, 65.3a) on the retentate side (65, 65.1, 65.2, 65.3) and a line (75a, 75.1a, 75.2a, 75.3a) on the filtrate side (75, 75.1, 75.2, 75.3);
b) filling the virus filtration module(s) (10, 10.1, 10.2, 10.3) and the connected lines with liquid (70) at a constant pressure of at most 98±5 kPa, with the following steps:
b1) filling all lines connected to the virus filtration module(s) (10, 10.1, 10.2, 10.3) with liquid (70), but without filling the virus filtration module(s) (10, 10.1, 10.2, 10.3);
b2) filling the inner region (35) of the hollow fibers (50.1, 50.2, 50.1a, 50.1b) of the virus filtration module(s) (10, 10.1, 10.2, 10.3) with liquid (70), wherein the line (55a, 55.1a, 55.2a, 55.3a) on the feed side (55, 55.1, 55.2, 55.3) and the line (65a, 65.1a, 65.2a, 65.3a) on the retentate side (65, 65.1, 65.2, 65.3) are opened and the line (75a, 75.1a, 75.2a, 75.3a) on the filtrate side (75, 75.1, 75.2, 75.3) is closed and
b3) filling the interstices (38.1, 38.2) of the hollow fibers (50.1, 50.2, 50.1a, 50.1b) of the virus filtration module(s) (10, 10.1, 10.2, 10.3) with liquid (70), wherein in each case either the line (55a, 55.1a, 55.2a, 55.3a) on the feed side (55, 55.1, 55.2, 55.3) and the line (75a, 75.1a, 75.2a, 75.3a) on the filtrate side (75, 75.1, 75.2, 75.3) are opened and the line (65a, 65.1a, 65.2a, 65.3a) on the retentate side (65, 65.1, 65.2, 65.3) are closed or the line (65a, 65.1a, 65.2a, 65.3a) on the retentate side (65, 65.1, 65.2, 65.3) and the line (75a, 75.1a, 75.2a, 75.3a) on the filtrate side (75, 75.1, 75.2, 75.3) are opened and the line (55a, 55.1a, 55.2a, 55.3a) on the feed side (55, 55.1, 55.2, 55.3) is closed;
wherein, in steps b2) and b3), the liquid (70) is fed to the virus filtration module(s) (10, 10.1, 10.2, 10.3) from the line (55a, 55.1a, 55.2a, 55.3a) on the feed side (55, 55.1, 55.2, 55.3) or from the line (65a, 65.1a, 65.2a, 65.3a) on the retentate side (65, 65.1, 65.2, 65.3);
c) stopping the feed of liquid (70) as soon as the virus filtration module(s) (10, 10.1, 10.2, 10.3) and the lines are filled with liquid (70), wherein the pressure is maintained, and closing all lines;
d) feeding air (80) to the virus filtration module(s) (10, 10.1, 10.2, 10.3) with step d1) or d2):
d1) opening in each case the line (55a, 55.1a, 55.2a, 55.3a) on the feed side (55, 55.1, 55.2, 55.3) and the line (75a, 75.1a, 75.2a, 75.3a) on the filtrate side (75, 75.1, 75.2, 75.3) of the virus filtration module(s) (10, 10.1, 10.2, 10.3) and in each case leaving the line (65a, 65.1a, 65.2a, 65.3a) on the retentate side (65, 65.1, 65.2, 65.3) closed, wherein the pressure is maintained in order to feed air (80) with a constant pressure at the same level as the liquid pressure from the line (55a, 55.1a, 55.2a, 55.3a) on the feed side (55, 55.1, 55.2, 55.3) into the virus filtration module(s) (10, 10.1, 10.2, 10.3);
or
d2) opening in each case the line (65a, 65.1a, 65.2a, 65.3a) on the retentate side (65, 65.1, 65.2, 65.3) and the line (75a, 75.1a, 75.2a, 75.3a) on the filtrate side (75, 75.1, 75.2, 75.3) of the virus filtration module(s) (10, 10.1, 10.2, 10.3) and in each case leaving the line (55a, 55.1a, 55.2a, 55.3a) on the feed side (55, 55.1, 55.2, 55.3) closed, wherein the pressure is maintained in order to feed air (80) with a constant pressure at the same level as the liquid pressure from the line (65a, 65.1a, 65.2a, 65.3a) on the retentate side (65, 65.1, 65.2, 65.3) into the virus filtration module(s) (10, 10.1, 10.2, 10.3);
wherein during step d1) or d2) the volume flow of the air (80) through the one or more virus filtration modules (10, 10.1, 10.2, 10.3) is measured;
e) automatically detecting a chain of air bubbles (130) occurring in the line (75a, 75.1a, 75.2a, 75.3a) on the filtrate side (75, 75.1, 75.2, 75.3) of the virus filtration module(s) (10, 10.1, 10.2, 10.3); and
optionally, in a step f) following step e), an evaluation, in particular an automatic evaluation, of the integrity test is carried out, wherein the test is evaluated as passed if during the test no continuous chain of air bubbles is detected for a duration of at least 20 seconds in the line (75a, 75.1a, 75.2a, 75.3a) on the filtrate side (75, 75.1, 75.2, 75.3), wherein in particular a counting apparatus for the air bubbles of the chain of air bubbles (130) is used, and at the same time a volume flow of the air (80) through the virus filtration modules (10, 10.1, 10.2, 10.3) of at most 8 mL/min per virus filtration module (10, 10.1, 10.2, 10.3) is measured.

2. The method according to claim 1,
characterized in that
the automatic detection of an occurring chain of air bubbles (130) in step e) is carried out by optical means, in particular a scattered light sensor (120) is used, wherein the line (75a, 75.1a, 75.2a, 75.3a) on the filtrate side (75, 75.1, 75.2, 75.3) for optical detection is selected so that it consists of or comprises transparent material.

3. The method according to claim 1,
characterized in that
after carrying out step c), the liquid (70) present is completely emptied from the inner region (35) of the hollow fibers (50.1, 50.2, 50.1a, 50.1b) and the connected line (55a, 55.1a, 55.2a, 55.3a)

4. The method according to claim 1,
characterized in that
wherein the volume flow of air (80) through the one or more virus filtration modules (10, 10.1, 10.2, 10.3) is measured only during step e).

5. The method according to claim 1,
characterized in that
in a step f following step e an evaluation, in particular an automatic evaluation, of the integrity test is carried out, wherein the test is evaluated as passed if during the test no continuous chain of air bubbles is detected for a duration of at least 20 seconds in the line (75a, 75.1a, 75.2a, 75.3a) on the filtrate side (75, 75.1, 75.2, 75.3), wherein in particular a counting apparatus for the air bubbles of the chain of air bubbles (130) is used, and at the same time a volume flow of the air (80) through the virus filtration modules (10, 10.1, 10.2, 10.3) of at most 8 mL/min per virus filtration module (10, 10.1, 10.2, 10.3) is measured.

6. The method according to claim 1,
characterized in that
the liquid (70) according to step b2) and b3) is fed to the virus filtration module(s) (10, 10.1, 10.2, 10.3) from the line (55a, 55.1a, 55.2a, 55.3a) on the feed side (55, 55.1, 55.2, 55.3) and the air (80) according to step d2) is fed to the virus filtration module(s) (10, 10.1, 10.2, 10.3) from the line (65a, 65.1a, 65.2a, 65.3a) on the retentate side (65, 65.1, 65.2, 65.3).

7. The method according to claim 1,
characterized in that
in steps b2) and b3), the filling level of the virus filtration module(s) (10, 10.1, 10.2, 10.3) is monitored by:
measuring the pressure prior to and after each virus filtration module (10, 10.1, 10.2, 10.3) and determining the differential pressure and/or
determining the amount of liquid (70) fed in relation to the dead volume of the filtration apparatus (100), including each virus filtration module (10, 10.1, 10.2, 10.3), wherein: the amount of liquid (70) fed > dead volume.

8. The method according to claim 1,
characterized in that
steps b) to e) and optionally f) are carried out with the support of a computer program in the form of a process control system, wherein in particular the process control system is selected so that it can also support the carrying out of virus filtration.

9. The method according to claim 1,
characterized in that
the method for testing the integrity of a virus filtration module (10, 10.1, 10.2, 10.3) according to steps a) to e) and optionally f) is carried out twice and between the two methods a virus filtration is effected with the tested virus filtration module(s) (10, 10.1, 10.2, 10.3).

10. The method according to claim 1,
characterized in that
the liquid (70) is selected from distilled water, bi-distilled or deionized water, ultrapure water or sterile water, in particular water for injection purposes or a buffer solution including water, and/or the air (80) is selected from process air.

11. The method according to claim 1,
characterized in that
prior to the entry of the liquid (70) into each virus filtration module (10, 10.1, 10.2, 10.3) in steps b1) and b2), the liquid (70) is passed through an inlet filter in order to filter out possible impurities and contamination from the liquid (70) and/or
after the exit of the liquid (70) from each virus filtration module (10, 10.1, 10.2, 10.3) in steps b2) and b3), the liquid (70) is passed through an outlet filter and/or
the air (80) is passed through an air filter (85) prior to entry into each virus filtration module (10, 10.1, 10.2, 10.3).

12. The method according to claim 1,
characterized in that
during step d) and prior to step e), a stabilization period of up to 600 s is observed, during which the air (80) is further dispersed.

13. The method according to claim 1,
characterized in that
transparent portions in the form of windows (78.1, 78.2) are provided in the line (75a, 75.1a, 75.2a, 75.3a) on the filtrate side (75, 75.1, 75.2, 75.3) for detection by optical means, wherein the windows (78.1, 78.2) are made of transparent plastics material, in particular selected from polystyrene (PS), polymethyl methacrylate (PMMA) or polycarbonate (PC), polyvinyl chloride (PVC), polyphenylene ether (PPO) or polyethylene (PE) or mixtures thereof; or selected from glass of hydrolytic class 1 or sapphire glass.

14. The method according to claim 1,
characterized in that
in the case of detection by optical means, the detection apparatus (120) is arranged on a line (75a) running from bottom to top, so that the chain of air bubbles (130) to be detected can rise in the line (75a).

15. The method according to claim 1,
characterized in that
an air-conducting line (85a) leading from the air filter (85) is provided with a portion that has the highest point of the filtration apparatus (100), so that no liquid (70) can rise to the air filter (85).

16. The method according to claim 1,
characterized in that
when testing a virus filtration module (10, 10.1, 10.2, 10.3), it is arranged so that the hollow fibers (50.1, 50.2, 50.1a, 50.1b) run in a horizontal direction or in a vertical direction or in an intermediate position between horizontal and vertical directions,
and when testing a plurality of virus filtration modules (10.1, 10.2, 10.3) simultaneously, they are arranged so that the hollow fibers (50.1, 50.2, 50.1a, 50.1b) are aligned parallel to one another, in particular run in the vertical direction or in an intermediate position between the horizontal and vertical directions.

17. The method according to claim 1,
characterized in that
when testing a plurality of virus filtration modules (10.1, 10.2, 10.3) simultaneously, the liquid (70) and/or the air (80) is fed in each case via a line (55.1a, 55.2a, 55.3a or 65.1a, 65.2a, 65.3a) assigned to each virus filtration module (10.1, 10.2, 10.3) and from the same side for all virus filtration modules (10.1, 10.2, 10.3) to be tested and/or the respective lines (75.1a, 75.2a, 75.3a) on the filtrate side (75.1, 75.2, 75.3) are combined to form one line (75a), and the detection apparatus (120) is arranged on the combined line (75a).

18. (canceled)

19. The method according to claim 1,
characterized in that
prior to carrying out step a), sterilization, in particular hot steam sterilization, of the lines is carried out and in particular after sterilization of the lines and/or prior to carrying out step a), the lines are placed under an overpressure of approximately 100 kPa and subsequently a pressure reduction to a pressure P≤10 kPa is undertaken in all lines and/or prior to carrying out step d), a leak test is carried out on the filtration system (100) and the installed virus filtration module(s) (10.1, 10.2, 10.3) and/or
if a defect is detected in the integrity test during simultaneous testing of a plurality of virus filtration modules (10.1, 10.2, 10.3), an individual test of the virus filtration modules (10.1, 10.2, 10.3) is carried out according to steps b) to e) and optionally f), the defective virus filtration module(s) (10.1, 10.2, 10.3) is/are replaced and the method according to steps b) to e) and optionally f) is repeated with the replaced virus filtration module(s) or with all virus filtration modules (10.1, 10.2, 10.3).

20. (canceled)

21. The method according to claim 1,
characterized in that
the filtration apparatus is designed so that virus filtration can be carried out therewith, wherein the virus filtration is carried out at the same or a lower pressure selected according to claim 1.

22. (canceled)

23. A filtration apparatus (100) for carrying out the method according to claim 1 preceding, comprising
one or more virus filtration modules (10, 10.1, 10.2, 10.3) which are in the form of hollow fiber filter modules, in each case comprising hollow fibers (50.1, 50.2, 50.1a, 50.1b) having an inner region (35) and hollow fiber interstices (38.1, 38.2), in each case having a feed side (55, 55.1, 55.2, 55.3), a retentate side (65, 65.1, 65.2, 65.3) and a filtrate side (75, 75.1, 75.2, 75.3);
devices for attaching and fastening one or more virus filtration modules (10, 10.1, 10.2, 10.3);
in each case, one line (55a, 55.1a, 55.2a, 55.3a) on the feed side (55, 55.1, 55.2, 55.3), one line (65a, 65.1a, 65.2a, 65.3a) on the retentate side (65, 65.1, 65.2, 65.3) and one line (75a, 75.1a, 75.2a, 75.3a) on the filtrate side (75, 75.1, 75.2, 75.3) of each virus filtration module (10, 10.1, 10.2, 10.3) for feeding liquid (70) and/or air (80) at a constant pressure of at most 98±5 kPa to the virus filtration module(s) (10, 10.1, 10.2, 10.3);
opening and closing devices (55v, 55.1v, 55.2v, 55.3v, 65v, 65.1v, 65.2v 65.3v, 75v, 75.1v, 75.2v, 75.3v, 75av, 75bv, 85v, 85.1v, 85.2v) for the respective lines;
a connector for feeding liquid (70) at a constant pressure of at most 98±5 kPa having the lines to the virus filtration module(s) (10, 10.1, 10.2, 10.3);
a connector for feeding air (80) of a constant pressure at the same level as the liquid pressure having the lines to the virus filtration module(s) (10, 10.1, 10.2, 10.3);
a detection apparatus (120) for automatically detecting a chain of air bubbles (130) in the line (75a) on the filtrate side (75, 75.1, 75.2, 75.3) of the virus filtration module(s) (10, 10.1, 10.2, 10.3) and
a measuring apparatus for measuring the volume flow of air (80) through the virus filtration module(s) (10, 10.1, 10.2, 10.3).

24. The filtration apparatus (100) according to claim 23,
characterized in that
the detection apparatus represents a scattered light sensor (120), wherein the line (75a, 75.1a, 75.2a, 75.3a) on the filtrate side (75, 75.1, 75.2, 75.3) for optical detection is selected so that it consists of or comprises transparent material.

25. The filtration apparatus (100) according to claim 23,
characterized in that
transparent portions in the form of windows (78.1, 78.2) are provided in the line (75a, 75.1a, 75.2a, 75.3a) on the filtrate side (75, 75.1, 75.2, 75.3) for detection by optical means, wherein the windows (78.1, 78.2) are made of transparent plastics material, in particular selected from polystyrene (PS), polymethyl methacrylate (PMMA) or polycarbonate (PC), polyvinyl chloride (PVC), polyphenylene ether (PPO) or polyethylene (PE) or mixtures thereof; or selected from glass of hydrolytic class 1 or sapphire glass.

26. The filtration apparatus (100) according to claim 23,
characterized in that
in the case of detection by optical means, the detection apparatus (120) is arranged on a line (75a) running from bottom to top, so that the chain of air bubbles (130) can rise in the line (75a), and/or an air-conducting line (85a) leading from the air filter (85) has a portion that represents the highest point of the filtration apparatus (100), so that no liquid (70) can rise to the air filter (85), and/or the respective lines (75.1a, 75.2a, 75.3a) on the filtrate side (75.1, 75.2, 75.3) are combined to form a line (75a), and the detection apparatus (120) is arranged on the combined line (75a).

27. The filtration apparatus (100) according to claim 23,
characterized in that
if a plurality of virus filtration modules (10.1, 10.2, 10.3) are present in the filtration apparatus (100) simultaneously, they are arranged so that the hollow fibers (50.1, 50.2, 50.1a, 50.1b) of the virus filtration modules (10.1, 10.2, 10.3) are in each case aligned parallel to one another, in particular run in the vertical direction or are aligned in an intermediate position between the horizontal and vertical directions.

28. The filtration apparatus (100) according to claim 23
characterized in that
the filtration apparatus (100) for carrying out the method for testing the integrity of a virus filtration module (10, 10.1, 10.2, 10.3) according to claim 1 is designed so that virus filtration can be carried out therein with the virus filtration module(s) (10, 10.1, 10.2, 10.3) being tested.

29. A method for cultivating prokaryotic or eukaryotic cells in liquid cell culture in a bioreactor to produce a product, in particular a protein, and carrying out virus filtration of the product obtained using one or more virus filtration modules (10, 10.1, 10.2, 10.3), wherein the method for testing the integrity of the one or more virus filtration modules (10, 10.1, 10.2, 10.3) according to claim 1 is carried out prior to and/or after virus filtration.

30. A method for producing a recombinant protein, wherein the method comprises the following steps:
step a) cultivating prokaryotic or eukaryotic cells expressing a recombinant protein in cell culture in a bioreactor;
step b) harvesting the recombinant protein;
step c) purifying the recombinant protein;
step d) removing viruses from the recombinant protein obtained in a virus filtration with one or more virus filtration modules (10, 10.1, 10.2, 10.3), wherein the method for testing the integrity of the one or more virus filtration modules (10, 10.1, 10.2, 10.3) according to claim 1 is carried out prior to and/or after virus filtration.
